(19) United States
(12) Patent Application Publication
(10) Pub. No.: US 2008/0213871 A1
(43) Pub. Date: Sep. 4, 2008

(54) Altering regulation of maize lignin biosynthesis enzymes via RNAi technology

(75) Inventor: Masomeh B. Sticklen, East Lansing, MI (US)

Correspondence Address: Peter G. Carroll;MEDLEN & CARROLL, LLP, Suite 350, 101 Howard Street, San Francisco, CA, 94105, US

(73) Assignee: Michigan State University

(21) Appl. No.: 11/998,247

(22) Filed: Nov. 29, 2007

Related U.S. Application Data
(60) Provisional application No. 60/872,205, filed on Dec. 1, 2006.

(51) Int. Cl. D21C 1/00; C12N 15/82; C12N 5/10

(52) U.S. Cl. 435/277; 435/320.1; 435/412; 800/285

(57) ABSTRACT

The present invention relates to compositions and methods for providing RNA Interference (RNAi) vectors comprising maize lignin biosynthesis enzymes for altering lignin content of plants. Specifically, plants comprising RNAi maize lignin vectors for reducing or altering lignin content are provided for reducing pretreatment costs of biofuel production. Additionally, RNAi maize lignin vectors are provided for altering cellulose production in plants for reducing pretreatment costs of plant biomass processing by increasing amounts of fermentable sugars.

### FIELD OF THE INVENTION

The present invention relates to compositions and methods for providing RNA Interference (RNAi) vectors comprising maize lignin biosynthesis enzymes for altering lignin content and lignin residue structural compositions of plants. Specifically, plants comprising RNAi maize lignin vectors for reducing or altering lignin content and chemical compositions are provided for reducing pretreatment costs of biofuel production. Additionally, RNAi maize lignin vectors are provided for altering cellulose production in plants for reducing pretreatment costs of plant biomass processing by increasing amounts of fermentable sugars.

### BACKGROUND OF THE INVENTION

Plant lignocellulosic biomass is renewable, cheap and globally available at least in 10-50 billion tons per year. At present, plant biomass is converted to fermentable sugars for the production of biofuels using pretreatment processes that disrupt the lignocellulose complexes in order to remove the lignin, thus allowing the access of microbial enzymes for cellulose deconstruction.

The operation costs of standard pretreatments for removing or altering lignin by adding ammonia, acid and/or heat treat of the feedstock plant biomass add about $1.15 to $2.25/gallon of the final ethanol product (Eggeman, 2005, Bioresource Technology, 96(18):2019-2025, herein incorporated by reference). These additional costs do not include the production and use of hydrolytic enzymes, the process of fermentation of sugars into alcohol fuel; or feedstock biomass production, transportation and storage. Therefore, lignin is considered the costly blocking agent in conversion of biomass into alcohol fuels (Sticklen, 2006, Current Opin. Biotech. 17(3):315-319; Sticklen, 2006, Category Session 1B: Plant Biotechnology and Genomics (2006), 2007, Applied Biochemistry and Biotechnology, 137-140(1-12):205, abstract, herein incorporated by reference). Further, both the pretreatments and the production of enzymes in microbial tanks are expensive.

Currently, ethanol produced in the United States is primarily derived from the starch of maize seeds or kernels. If all maize kernels currently produced in the U.S. were used for ethanol production, they would merely provide a total of 15% of the U.S. transportation fuels (Sticklen, 2007, Crop Sci. 47:2238-2248, herein incorporated by reference). However, the United States Government has set a goal to produce one billion tons of biomass for conversion into ethanol in order to supply 30% of the transportation fuels by 2030 (USDA-DOE, 2007, herein incorporated by reference).

This millions of tons of crop biomass will be corn lignocellulosic biomass (leaves, stalks, inner portion of maize kernels, and outer portions of corn kernels). About 17-20% of maize biomass is lignin, a blocking agent to the pretreatment processes that produces ethanol (Sticklen, 2007, Crop Sci. 47:2238-2248, herein incorporated by reference).

Thus, what is needed are compositions and methods for reducing and/or modifying the lignin in plants, in particular maize kernels, destined as sources of plant biomass for ethanol biofuel production. In particular, there is a need to modify lignin at a level where it does not interfere with the plant structural integrity or reduce a plant's defense against insects and pathogens.

### SUMMARY OF THE INVENTION

The present invention relates to compositions and methods for providing RNA Interference (RNAi) vectors comprising maize lignin biosynthesis enzymes for altering lignin content and/or composition of plants. Specifically, plants comprising RNAi maize lignin vectors for reducing or altering lignin are provided for reducing pretreatment costs of biofuel production. Additionally, RNAi maize lignin vectors are provided for increasing cellulose production in plants. Because cellulose is the main source of polysaccharides, an increase in cellulose means an increase in the amounts of fermentable sugars.

The invention provides an RNAi expression vector for silencing a gene in a cell. In particular for silencing genes for altering lignin biosynthesis. In some embodiments, the silenced gene reduces lignin content of a plant cell. In some embodiments, the silenced gene reduces lignin content of a plant tissue. In a further embodiment, the silencing genes result in an increase of production of cell wall polysaccharides. In a further embodiment, increase of production of cell wall polysaccharides is an increase in cellulose biosynthesis. The present invention is not limited by the type of gene silencing.

The present invention is not limited by the type of gene target to be silenced. The present invention is not limited by the type of silenced gene. In particular, the present invention is not limited to target genes for lignin. In particular, the present invention is not limited to target genes for cellulose biosynthesis. In particular, the present invention is not limited to target genes for hemicellulose biosynthesis. In preferred embodiments, the silenced gene is a gene target.

The invention provides an expression vector, said vector comprising a first nucleotide that interferes with a second nucleotide encoding a polypeptide for altering lignin biosynthesis. The present invention is not limited by the type of lignin biosynthesis. In some embodiments, the altering lignin biosynthesis is reducing lignin biosynthesis. In some embodiments, the altering lignin biosynthesis is modifying the chemical compositions of plant lignin residues. In some embodiments, the altering lignin biosynthesis is altering cellulose biosynthesis. In some embodiments, the altering lignin biosynthesis is altering hemicellulose biosynthesis. In some embodiments, the altering lignin biosynthesis is increasing cellulose biosynthesis. In some embodiments, the altering lignin biosynthesis is increasing hemicellulose biosynthesis. The present invention is not limited by the lignin, cellulose or hemicellulose gene target. In some embodiments, the gene is a plant biosynthesis gene. In some embodiments, the gene is a plant biosynthesis gene. In some embodiments, the gene is a maize gene. The present invention is not limited by the type of RNAi molecule. In some embodiments, the RNAi molecule comprises a sequence encoding an siRNA, hairpin siRNA, miRNA, and snRNA. The present invention is not limited by the type of silenced gene. In some embodiments, the second nucleotide comprises SEQ ID NOs: 1-5. In some embodiments, the polypeptide comprises SEQ ID NOs: 6-10. In some embodiments, the vector further comprises a sequence that permits inducible expression of said RNAi molecule.

The invention further provides a composition comprising a host cell comprising an expression vector, said vector comprising a first nucleotide that interferes with a second nucleotide encoding a polypeptide for altering lignin and/or cellulose biosynthesis. The present invention is not limited by the type of host cell. In some embodiments, the cell is in culture. In some embodiments, the cell resides in vivo. In some embodiments, the cell resides in vitro. In some embodiments, the cell comprises a host tissue. In some embodiments, the vector is stably integrated into the genome of said cell. In some embodiments, the host cell is a maize cell. In some embodiments, the maize cell resides in a plant part. The present invention is not limited by the type of plant part. In some embodiments, the plant part is a kernel part, leaf part, stem part or a whole plant.

The invention further provides a method for gene expression silencing, comprising the step of transfecting a cell with an expression vector, said vector comprising a first nucleotide that interferes with a second nucleotide encoding a polypeptide for altering lignin and/or cellulose biosynthesis.

In some embodiments, the present invention provides transgenic plants comprising heterologous nucleic acid sequences encoding a double stranded nematode RNA sequence, wherein said double stranded RNA sequence inhibits an enzyme in the lignin biosynthesis pathway. The present invention is not limited to any particular expression construct or construct design. Indeed, the use of a variety of constructs and designs are contemplated. In some embodiments, the heterologous nucleic acid sequences are operably linked to the same promoter. In other embodiments, the heterologous nucleic acid sequences are operably linked to separate or different promoter sequences. In still other embodiments, the heterologous nucleic acid sequences are separated by a loop sequence. In some embodiments, the promoter is a tissue specific promoter, while in other embodiments the promoter is a constitutive promoter. The present invention is not limited to the use of any particular heterologous nucleic acid sequence. Indeed, the use of a variety of sequences is contemplated, including, but not limited to those that complementary to an RNA sequence selected from the group consisting of the enzymes identified in FIG. 1, and in lignin production, such as phenyl ammonia lyase (PAL; E.C. 4.3.1.5); cinnamate 4-hydroxylase (C4H; EC 1.14.13.11); C3H, para-coumarate 3-hydroxylase; S-adenosylmethione:caffeate/5-hydroxyferulate-O-methyltransferase (OMT, EC 2.1.1.6); caffeic acid O-methyltransferase (COMT, EC 2.1.1.68); caffeoyl-CoA O-methyltransferase CCoAOMT, EC 2.1.1.104); 4-coumarate:CoA ligase (4CL, EC 6.2.1.12); cinnamoyl-CoA reductase (CCR; EC 1.2.1.44); cinnamyl alcohol dehydrogenase (CAD, EC 1.1.1.195); SAD, sinapyl alcohol dehydrogenase; HCT, para-hydroxycinnamoyl-CoA:quinate shikimate para-hydroxycinnamoyltransferase; F5H: ferulate 5-hydroxylase; and members of their gene families. Likewise, the present invention is not limited to heterologous nucleic acid sequences of any particular length. Indeed, heterologous nucleic acid sequences of varying lengths may be utilized, including those from about 21 bases in length to the full length of the target RNA. In still further embodiments, the present invention provides plant tissue or material from the foregoing transgenic plants. The present invention is not limited to any particular tissue or material. Indeed, a variety of plant tissues and materials are contemplated. Accordingly, in some embodiments, the present invention provides seeds, leaves, roots, stalks or processed materials derived from the foregoing transgenic plants.

In some embodiments, the present invention provides vectors comprising heterologous nucleic acid sequences encoding a double stranded maize RNA sequence, wherein said double stranded RNA sequence inhibits an enzyme in the maize biosynthesis pathway. The present invention is not limited to any particular vector or vector design. Indeed, the use of a variety of vectors and designs are contemplated. In some embodiments, the heterologous nucleic acid sequences are operably linked to the same promoter. In other embodiments, the heterologous nucleic acid sequences are operably linked to separate or different promoter sequences. In still other embodiments, the heterologous nucleic acid sequences are separated by a loop sequence. In some embodiments, the promoter is a tissue specific promoter, while in other embodiments the promoter is a constitutive promoter. The present invention is not limited to the use of any particular heterologous nucleic acid sequence. Indeed, the use of a variety of sequences is contemplated, including, but not limited to those identified in FIG. 1. Likewise, the present invention is not limited to heterologous nucleic acid sequences of any particular length. Indeed, heterologous nucleic acid sequences of varying lengths may be utilized, including those from about 21 bases in length to the full length of the target RNA. In still further embodiments, the present invention provides a transgenic plant comprising the foregoing vectors. In other embodiments, the present invention provides animal feeds comprising plant tissue from the foregoing transgenic plants. In some embodiments, the plant tissue is selected from seeds and leaves. In still further embodiments, the present invention provides pharmaceutical compositions comprising materials derived from the foregoing transgenic plants.

In still further embodiments, the present invention provides methods of creating transgenic plants comprising transfecting a plant or plant tissue with the foregoing vector. In other embodiments, the present invention provides the transgenic plant produced by this process. In some embodiments, the methods further comprises harvesting the transgenic material and using the transgenic plant material to produce a pharmaceutical composition or animal feed. The present invention also provides the pharmaceutical compositions and animal feeds produced by these processes.

In still further embodiments, the present invention provides fermented plant material derived from the foregoing transgenic plants. In further embodiments, the present invention provides methods for producing ethanol comprising providing transgenic plant material comprising an RNAi vector as described above, and fermenting the plant material to produce ethanol.

The invention further provides an expression vector, wherein said vector comprises a first nucleotide, wherein said first nucleotide interferes with a second nucleotide encoding a polypeptide that alters lignin biosynthesis, and wherein said first nucleotide is in operable combination with a riburose-1,5-bisphosphate carboxylase small-subunit (RbcS1) promoter. In some embodiments, the vector expresses said first nucleotide in cytoplasm. In some embodiments, the promoter is a Chrysanthemum promoter. In some embodiments, the altered lignin biosynthesis reduced lignin biosynthesis. In some embodiments, the altered lignin biosynthesis modifies a lignin structure. In some embodiments, the altered lignin biosynthesis alters cellulose. In some embodiments, the altered lignin biosynthesis increases cellulose. In some embodiments, the altered lignin biosynthesis increases soluble cellulose. In some embodiments, the polypeptide is selected from the group consisting of a phenyl ammonia lyase; cinnamate 4-hydroxylase; para-coumarate 3-hydroxylase; S-adenosylmethione:caffeate/5-hydroxyferulate-O-methyltransferase caffeic acid O-methyltransferase; caffeoyl-CoA O-methyltransferase CCoAOMT; 4-coumarate:CoA ligase; cinnamoyl-CoA reductase; cinnamyl alcohol dehydrogenase; sinapyl alcohol dehydrogenase; para-hydroxycinnamoyl-CoA:quinate shikimate para-hydroxycinnamoyltransferase; ferulate 5-hydroxylase; homologs and orthologs thereof. In some embodiments, the second nucleotide is selected from the group consisting of SEQ ID NOs:1-5. In some embodiments, the polypeptide is selected from the group consisting of SEQ ID NOs:6-10. In some embodiments, the first nucleotide comprises an RNAi molecule selected from the group consisting of a siRNA, hairpin siRNA, miRNA and snRNA. In some embodiments, the expression vector further comprises an RNAi construct, wherein said construct comprises said first nucleotide sequence in an antisense direction in operable combination with said first nucleotide sequence in a sense direction.

The invention further provides a transgenic maize cell, wherein said maize cell comprises an RNAi gene silencing construct in operable combination with a riburose-1, 5-bisphosphate carboxylase small-subunit (RbcS1) promoter. In some embodiments, the RNAi construct comprises an oligonucleotide in an antisense direction in operable combination with said oligonucleotide in a sense direction. In some embodiments, the oligonucleotide comprises a portion of a first nucleotide sequence encoding a polypeptide selected from the group consisting of a phenyl ammonia lyase; cinnamate 4-hydroxylase; para-coumarate 3-hydroxylase; S-adenosylmethione:caffeate/5-hydroxyferulate-O-methyltransferase caffeic acid O-methyltransferase; caffeoyl-CoA O-methyltransferase CCoAOMT; 4-coumarate:CoA ligase; cinnamoyl-CoA reductase; cinnamyl alcohol dehydrogenase; sinapyl alcohol dehydrogenase; para-hydroxycinnamoyl-CoA:quinate shikimate para-hydroxycinnamoyltransferase; ferulate 5-hydroxylase; homologs and orthologs thereof. In some embodiments, the oligonucleotide comprises at least a portion of a second nucleotide sequence selected from the group consisting of SEQ ID NOs:1-5. In some embodiments, the maize cell comprises a plant part.

The invention further provides a composition comprising a transgenic maize cell, wherein said maize cell comprises an RNAi gene silencing construct in operable combination with a riburose-1,5-bisphosphate carboxylase small-subunit (RbcS1) promoter.

The invention further provides a method of gene silencing in a maize plant part, comprising, a) providing, i) a maize plant part, ii) a nucleic acid sequence encoding an enzyme, wherein said enzyme alters a lignin structure, iii) a gene silencing construct, wherein said RNAi gene silencing construct is in operable combination with a riburose-1,5-bisphosphate carboxylase small-subunit (RbcS1) promoter, and b) transfecting said gene silencing construct into said plant part for silencing said enzyme. In some embodiments, the silencing alters lignin production while retaining the desired characteristics of a plant cell wall.

The invention further provides a method for producing glucose from a lignocellulosic biomass, comprising, a) providing, i) a lignocellulosic biomass, comprising an RNAi gene silencing construct in operable combination with a riburose-1,5-bisphosphate carboxylase small-subunit (RbcS1) promoter, and ii) a composition capable of converting cellulose to glucose, b) converting said lignocellulosic biomass into glucose using said composition. In some embodiments, the lignocellulosic biomass comprises maize corn stover. In some embodiments, the composition is selected from the group consisting of an ammonia fiber and a hydrolytic enzyme.

### DESCRIPTION OF THE DRAWINGS

FIG. 1 shows an exemplary lignin biosynthesis pathway with lignin altering enzymes (A) old model (Humphreys and Chapple, 2002, herein incorporated by reference) and (B) revised model (Sticklen, 2007, Crop Sci. 47:2238-2248, herein incorporated by reference) Lignin biosynthesis pathway: PAL, phenyl ammonia lyase; C4H, cinnamate 4-hydroxylase; C3H, para-coumarate 3-hydroxylase; COMT, caffeic acid O-methyltransferase; CCoAOMT, caffeoyl-CoA O-methyltransferase; 4CL, 4-coumarate:CoA ligase; 4CL?? indicates that certain species have 4CL activity toward sinapic acid; CCR, cinnamoyl-CoA reductase; CAD, cinnamyl alcohol dehydrogenase; SAD, sinapyl alcohol dehydrogenase; HCT, para-hydroxycinnamoyl-CoA:quinate shikimate para-hydroxycinnamoyltransferase; CCR? And F5H? where ? indicates that enzymes whose substrates have not been tested; F5H: ferulate 5-hydroxylase; ? indicates that conversion has been demonstrated; ?? indicates that direct conversion not convincingly been demonstrated. ***, enzymatic assays in Arabidopsis have shown that the shikimate and quinate esters of paracoumaric acid are the ideal substrates for paracoumarate 3-hydroxylase (C3H). For example, in Arabidopsis, 4CL first converts the para-coumarate to para-coumaroyl-CoA, and then the C3H converts the para-coumaroyl-shikimate and para-coumaryoyl-quinate.

FIG. 2 shows exemplary maize lignin biosynthesis enzyme sequences.

FIG. 3 shows exemplary maize cDNA sequences encoding corn lignin biosynthesis Enzymes of the present inventions showing PCR primer sequences and amplified regions (A) Zea mays 4-coumarate coenzyme A ligase (4CL), (B) Zea mays cinnamoyl-CoA reductase (CCR), and (C) Zea mays cinnamyl alcohol dehydrogense (CAD). top—gene sequence, middle—PCR primers, forward (F) and reverse (R), (regions underlined above) and bottom—amplified regions.

FIG. 4 shows exemplary RNAi constructs of the present inventions.

FIG. 5 shows an exemplary RNAi vector, Impact Vector 1.1 comprising a RbcS1 Chrysanthemum promotor region.

FIG. 6 shows an exemplary transformation process providing transgenic corn plants of the present inventions produced in vitro after genetic transformation of multimeristems (see, B & C). A) DNA produced by E. coli and corn meristems, B) Maize multimeristem primordia produced for bombardment, C) Magnified multimeristem showing somatic embryos, D) Rooted shoot produced from B) in Phosphinotricin (PPT) selection, E) Plantlets transferred to soil and acclimated to the growth chamber and greenhouse conditions, F) Plants in growth chamber, and G) Mature corn plants growing in a greenhouse.

FIG. 7 shows exemplary transgenic events for integration of CCR_RNAi using polymerase chain reaction (PCR) assays for bar and the RNAi primer sequences (A) Northern blot and B) ethidium bromide stain of corresponding gel prior to RNA transfer.

FIG. 8 shows an exemplary Northern blot comparison of the transcription levels (expression) of an RNAi vector comprising bar in transgenic corn leaves with the transcription of the exemplary gene in wild type corn leaves. −Control: Water, +control: pDM302 construct, 1a through 1h and 3a through 3h represent RNAi transgenic plants.

### DEFINITIONS

To facilitate an understanding of the present invention, a number of terms and phrases are defined below:

The use of the article “a” or “an” is intended to include one or more. As used in this application, the singular form “a,” “an,” and “the” include plural references unless the context clearly dictates otherwise. For example, the term “an agent” includes a plurality of agents, including mixtures thereof.

The term “lignin” is used herein as a generic term that includes both lignins and lignocelluloses. More specifically, the term “lignin” refers to a heterogeneous complex of monomers and polymers in a mixture comprising para-hydroxyphenyl, (p-hydroxyphenyl), guaiacyl and syringyl residues and further comprising ether linkages and carbon-carbon linkages between monomers with extensive cross-links, such as hydroxycinnamic acid (i.e. p-coumaric acid and ferulic acid) bridges, to other cell wall polymers. For example, in grasses, lignin comprises monolignols para-coumaryl, coniferyl and sinapyl alcohol monomers. Lignin also refers to a polymer constructed of non-carbohydrate, alcohol units that are not fermentable, but must be separated from the cellulose and hemicellulose by chemical and other means for fermentation processes such as for producing ethanol biofuel.

The term “lignocellulosic” or “ligninocellulosic” or “lignocellulose” or “ligninocellulose” refers to a composite material of cellulose fibers embedded in a cross-linked matrix of rigid lignin while cellulose and hemicellulose bind the fibers. Lignocellulose plant structures also contain a variety of plant-specific chemicals in the matrix, such as extractives (for example, resins, phenolics, etc.), and minerals (for example, calcium, magnesium, potassium, etc.) that will leave ash when biomass is burned.

The term “lignocellulosic biomass” refers to a feedstock for biomass derived products, such as ethanol biofuel, for example, plant material, such as kernels parts or stems, and waste material of food processing or waste material from forest products industries that may be locally, readily, and abundantly available at low cost. The structural materials that plants produce to form the cell walls of kernels, leaves, stems or stalks, and woody portions of biomass are composed mainly of three polymers called cellulose, hemicellulose, and lignin.

The term “cellulose” refers to a very large polymer molecule composed of many hundreds or thousands of glucose molecules (i.e. polysaccharides). The molecular linkages in cellulose form linear chains that are rigid, highly stable, and resistant to chemical attack.

The term “hemicellulose” refers to short, highly branched, chains of sugars, such as five-carbon sugars (usually D-xylose and L-arabinose) and six-carbon sugars (D-galactose, D-glucose and D-mannose) and uronic acid.

The terms “altered lignin” and “altering lignin” refer to any changes in lignin biosynthesis, for example, a decreased or increased amount of lignin in a plant stem, leaf or a kernel, such as an amount of lignin reduced between 8% and 30% based on the location of a mutated enzyme in the lignin biosynthesis pathway (Chabbert, et al., 1994, J. Sci. Food. Agric. 64:349-355, herein incorporated by reference). Further, down-regulation of lignin or modification of lignin structure has been reported in several crops, except in corn, via down regulation of different enzymes involved with lignin biosynthesis pathway (Sticklen, 2007, Crop Science, 47: 2238-2248; Sticklen 2006, Current Opin. Biotech. 17(3):315-319, herein incorporated by reference). “Altering lignin” may also refer to a change in lignin structure, such as an alteration in lignin that would increase biomass production by decreasing pretreatment costs, for example, increasing cellulose would reduce the costs of pretreatment processes by increasing the level of fermentable sugars in corn biomass.

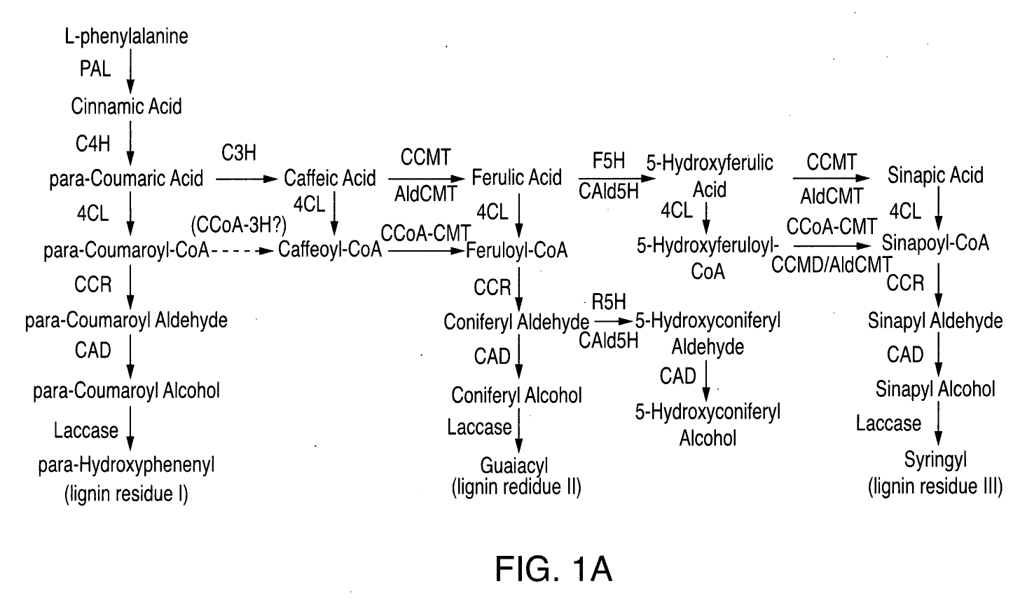

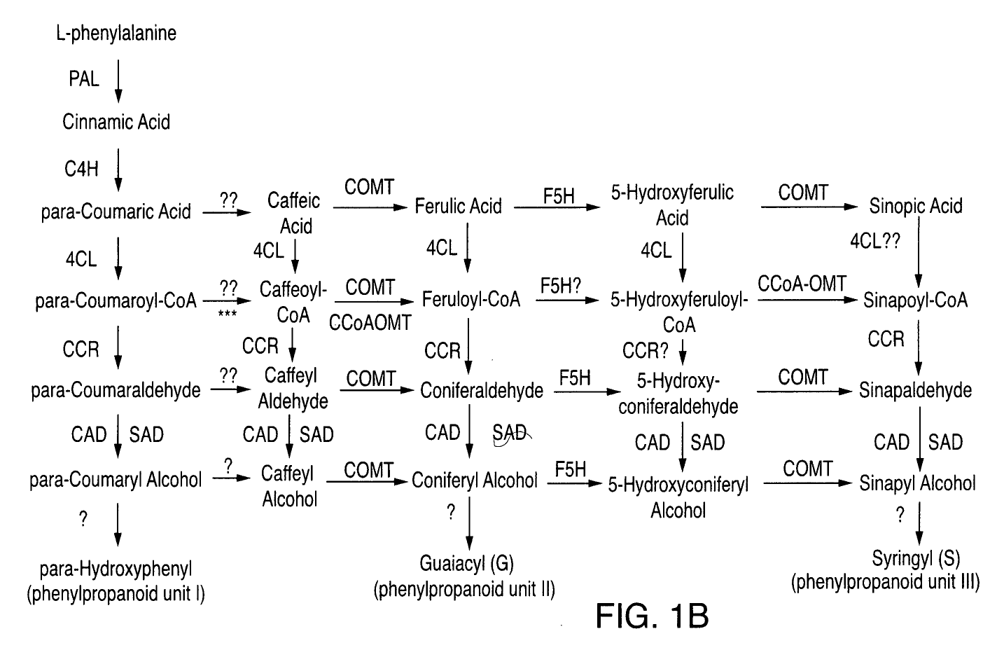

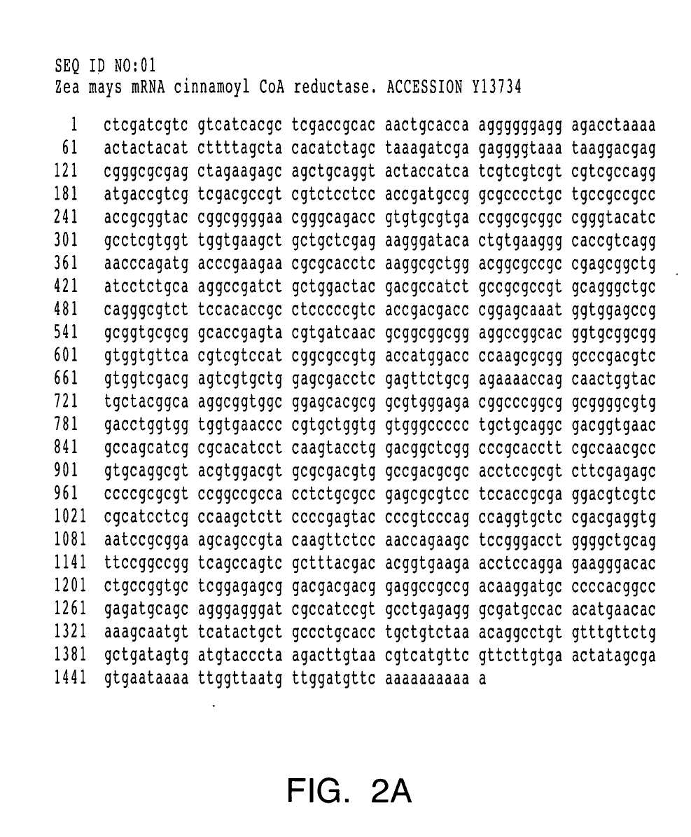

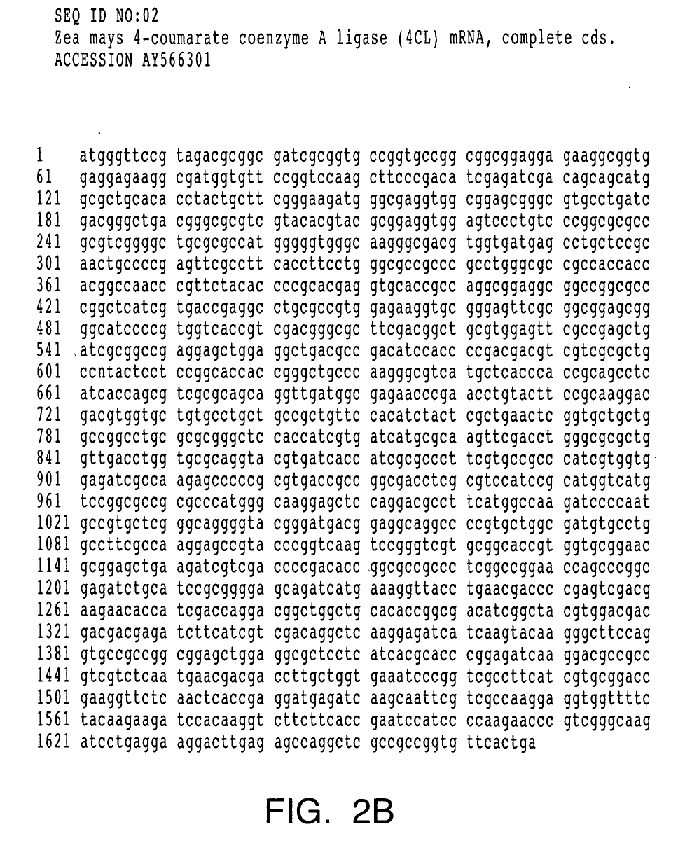

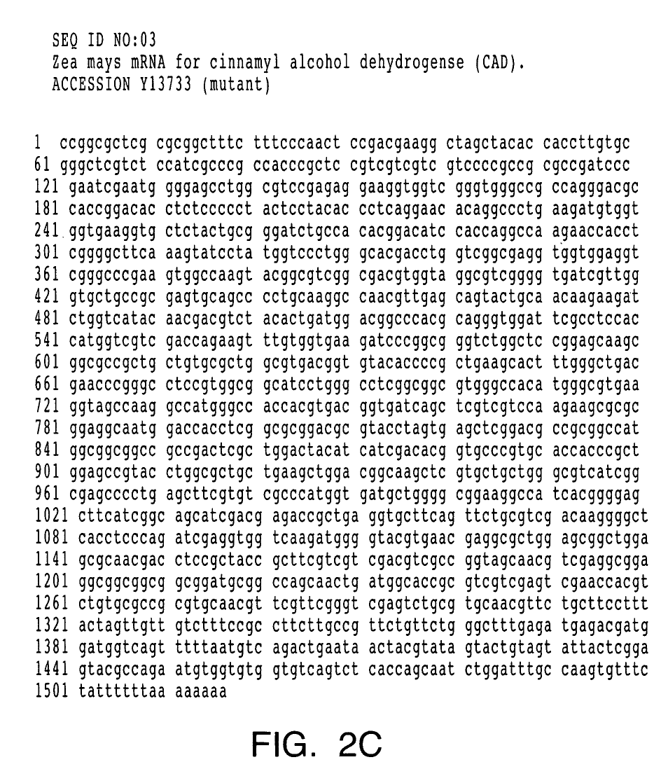

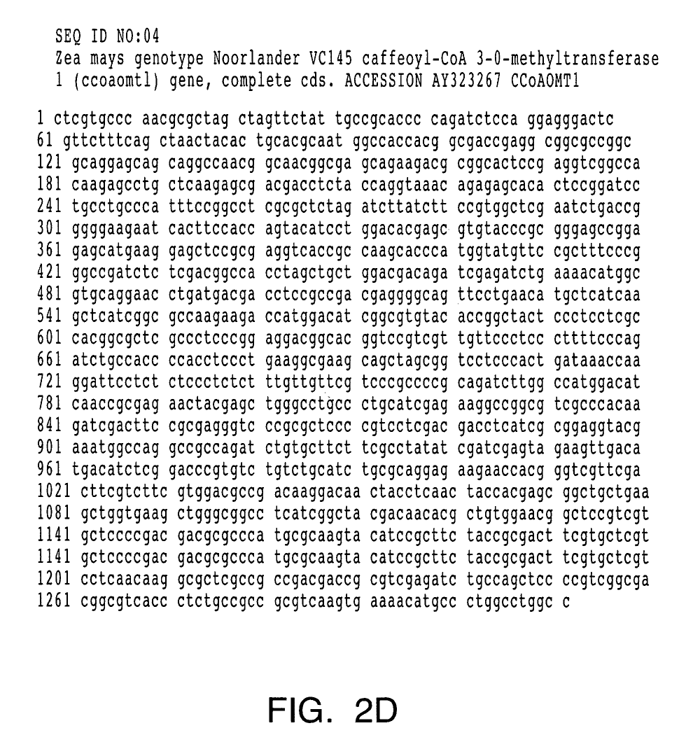

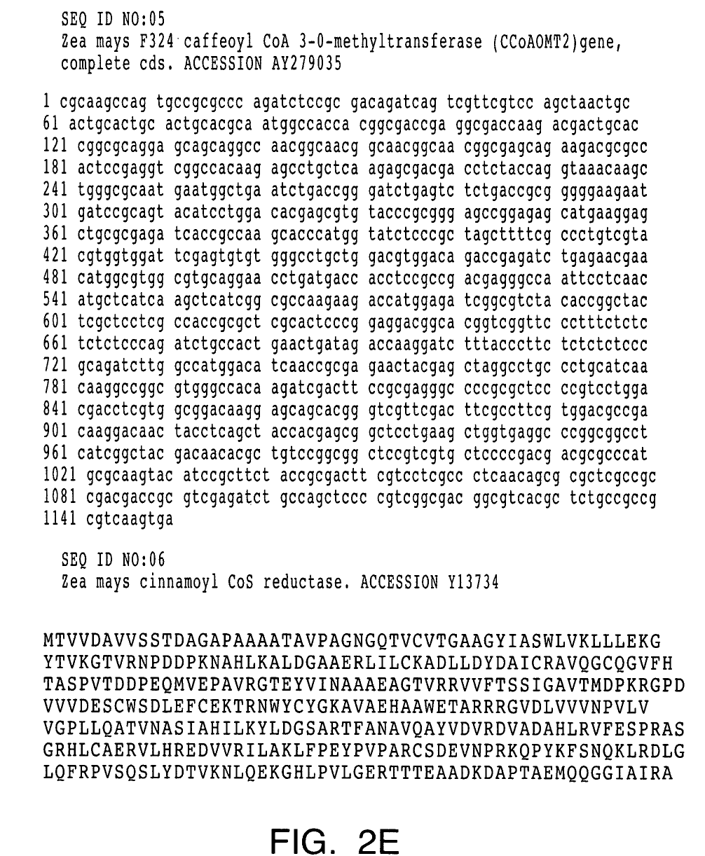

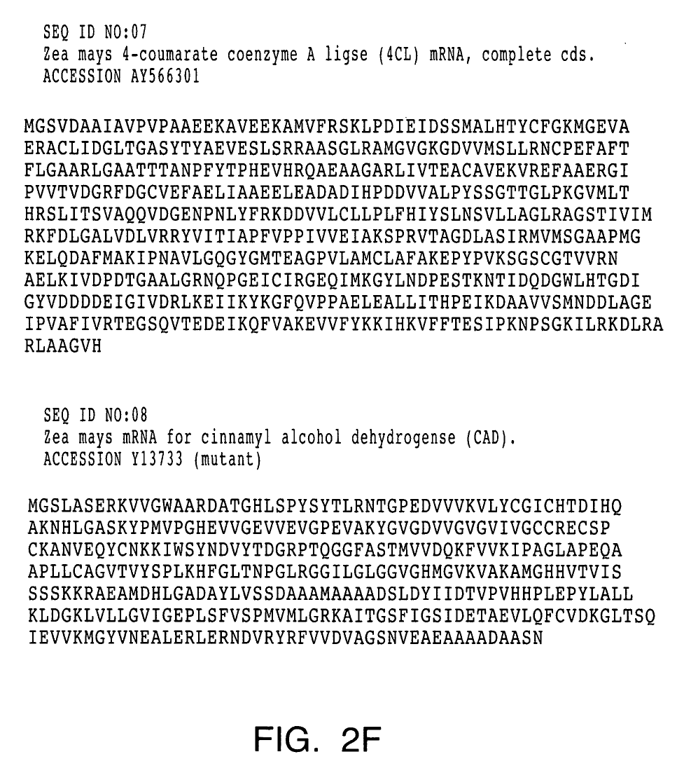

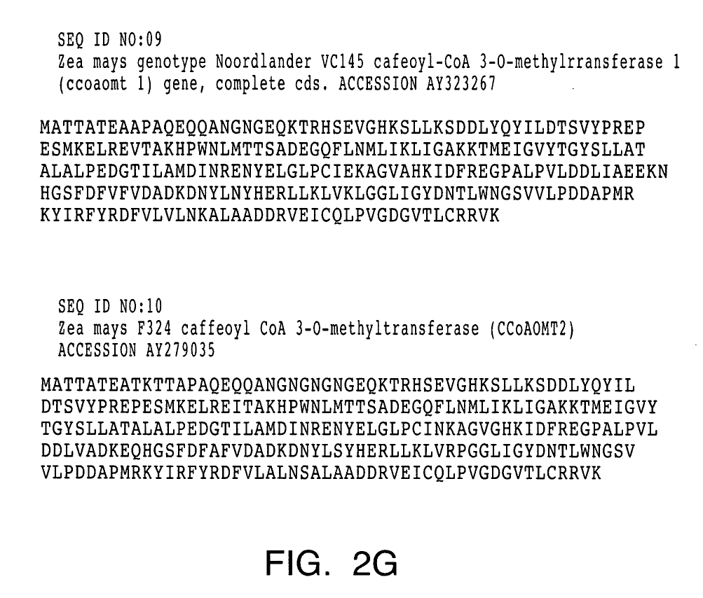

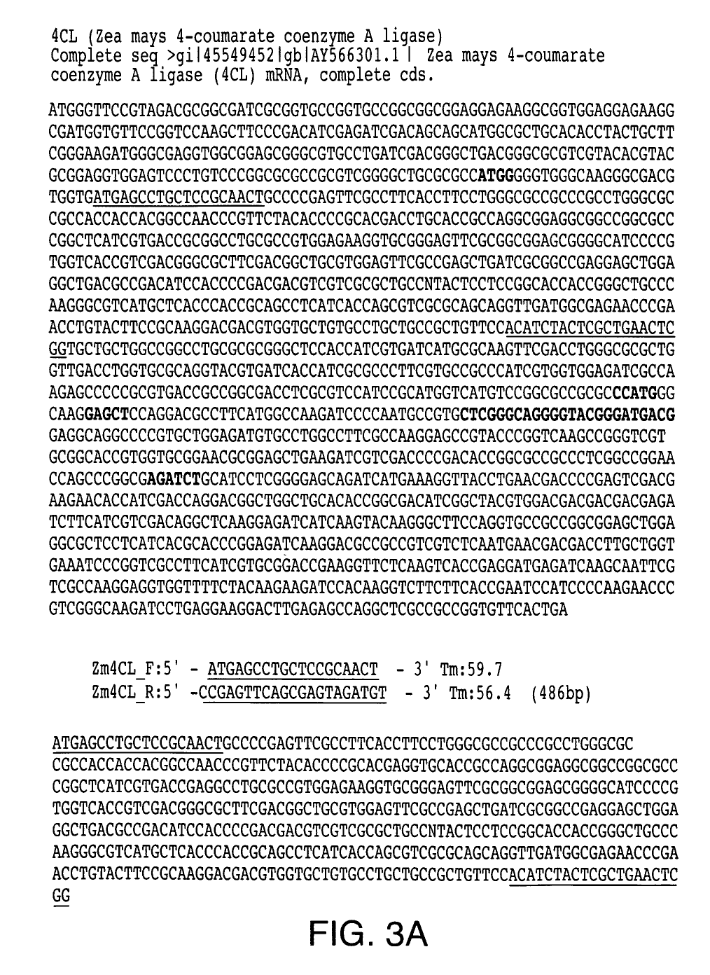

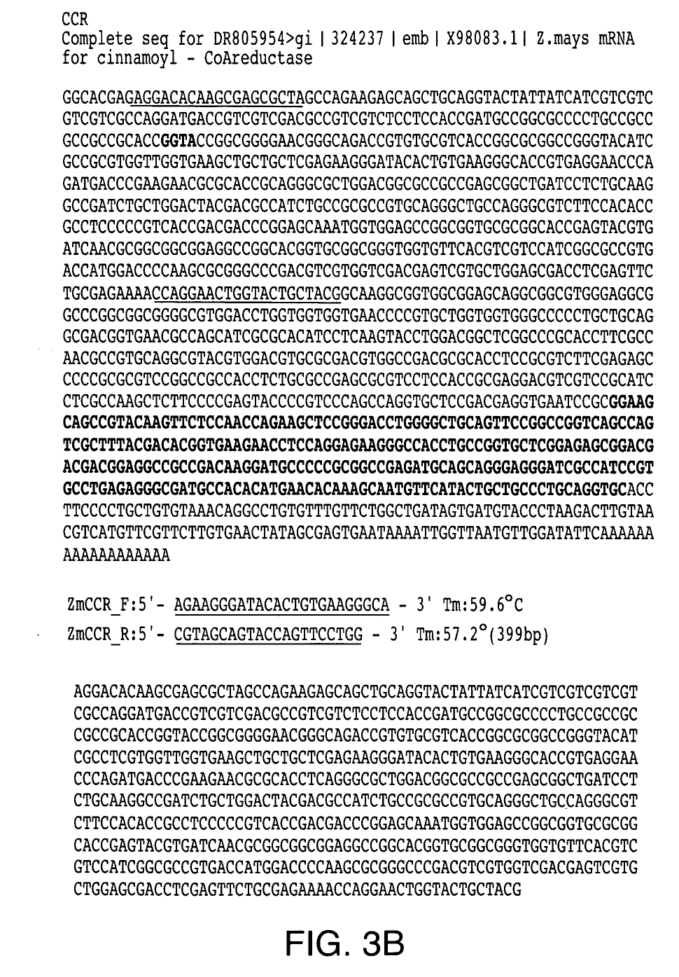

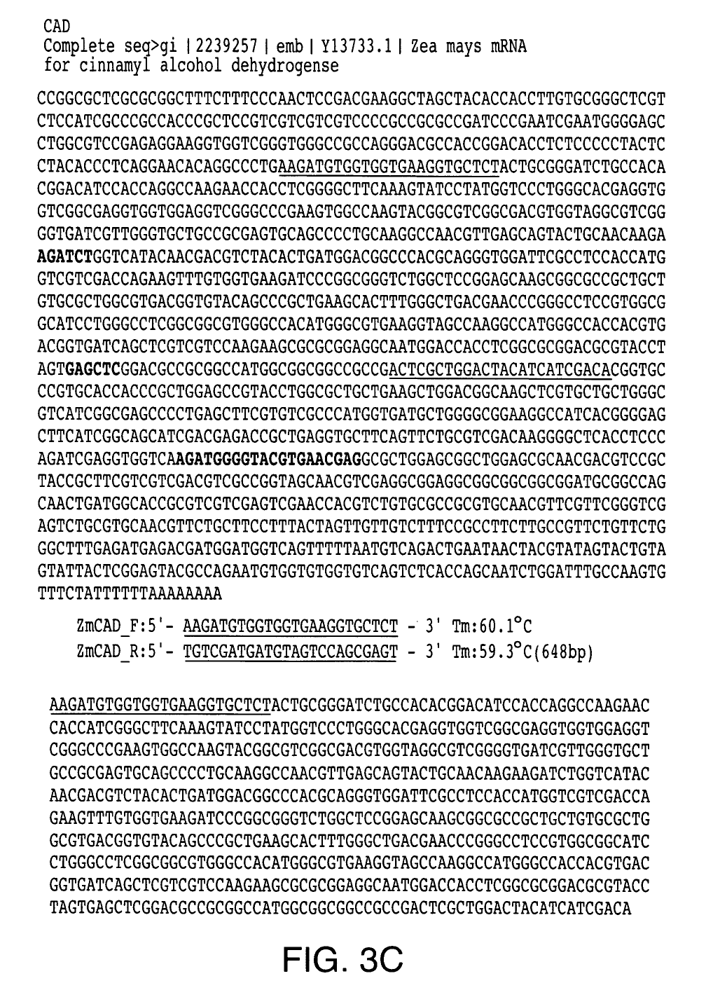

The term “modulate,” as used herein, refers to a change in the biological activity of a biologically active molecule. Modulation can be an increase or a decrease in activity, a change in binding characteristics, or any other change in the biological, functional, or immunological properties of biologically active molecules. For example, manipulation of each of the interconnected pathways of FIG. 1B is expected to modify or alter plant lignin (Sticklen, 2006, Current Opin. Biotech. 17(3):315-319; Ragauskas, et al., 200, Science 311:484-489, herein incorporated by reference).

As used herein, the term “altered levels” refers to the production of gene product(s) in transgenic organisms in amounts or proportions that differ from that of normal or non-transformed organisms.

The term “posttranscriptional gene silencing” or “PTGS” refers to silencing of gene expression in plants after transcription. PTGS may be gene specific or nongene specific, such that a group of related genes are silenced.

The term “overexpression” refers to the production of a gene product in transgenic organisms that exceeds levels of production in normal or non-transformed organisms.

The term “cosuppression” refers to the expression of a foreign gene that has substantial homology to an endogenous gene resulting in the suppression of expression of both the foreign and the endogenous gene.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing a mRNA-specific signal observed on Northern blots).

The term “recombinant DNA molecule” as used herein refers to a DNA molecule which is comprised of segments of DNA joined together by means of molecular biological techniques.

The term “recombinant protein” or “recombinant polypeptide” as used herein refers to a protein molecule which is expressed from a recombinant DNA molecule.

As used herein the term “portion” when in reference to a protein (as in “a portion of a given protein”) refers to fragments of that protein. The fragments may range in size from four amino acid residues to the entire amino acid sequence minus one amino acid.

As used herein the term “nucleic acid sequence” refers to an oligonucleotide, a nucleotide or a polynucleotide, and fragments or portions thereof, and vice versus, and to DNA or RNA of genomic or synthetic origin which may be single or double-stranded, and represent the sense or antisense strand. Similarly, “amino acid sequence” as used herein refers to peptide or protein sequence.

The term “antisense” when used in reference to DNA refers to a sequence that is complementary to a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex.

The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression.

“Ribozyme” refers to a catalytic RNA and includes sequence-specific endoribonucleases. “Antisense inhibition” refers to the production of antisense RNA transcripts capable of preventing the expression of the target protein.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “RNA interference” or “RNAi” refers to the silencing of a gene wherein the translation of a gene is down regulating or decreasing of gene expression by RNAi molecules (e.g., siRNAs, miRNAs). It is the process of sequence-specific, post-transcriptional gene silencing in animals and plants, initiated by RNAi molecules that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “ds siRNA” refers to a siRNA molecule that comprises two separate unlinked strands of RNA that form a duplex structure, such that the siRNA molecule comprises two RNA polynucleotides.

The term “hairpin siRNA” refers to a siRNA molecule that comprises at least one duplex region where the strands of the duplex are connected or contiguous at one or both ends, such that the siRNA molecule comprises a single RNA polynucleotide. The antisense sequence, or sequence which is complementary to a target RNA, is a part of the at least one double stranded region.

“MicroRNA molecules” (“miRNAs”) are small, noncoding RNA molecules that have been found in a diverse array of eukaryotes, including mammals. miRNA precursors share a characteristic secondary structure, forming short ‘hairpin’ RNAs. The term “miRNA” includes processed sequences as well as corresponding long primary transcripts (pri-miRNAs) and processed precursors (pre-miRNAs). Genetic and biochemical studies have indicated that miRNAs are processed to their mature forms by Dicer, an RNAse III family nuclease, and function through RNA-mediated interference (RNAi) and related pathways to regulate the expression of target genes (Hannon, 2002, Nature 418: 244-251; Pasquinelli et al. 2002, Annu. Rev. Cell. Dev. Biol. 18:495-513, all of which are herein incorporated by reference). miRNAs may be configured to permit experimental manipulation of gene expression in mammalian cells as synthetic silencing triggers ‘short hairpin RNAs’ (shRNAs) (Paddison et al. 2002, Cancer Cell 2:17-23, herein incorporated by reference). Silencing by shRNAs involves the RNAi machinery and correlates with the production of small interfering RNAs (siRNAs), which are a signature of RNAi.

The term, “microRNA molecules” or “miRNAs” refer to small, noncoding RNA molecules that have been found in a diverse array of eukaryotes, including plants. miRNA precursors share a characteristic secondary structure, forming short ‘hairpin’ RNAs. The term “miRNA” includes processed sequences as well as corresponding long primary transcripts (pri-miRNAs) and processed precursors (pre-miRNAs). Genetic and biochemical studies have indicated that miRNAs are processed to their mature forms by Dicer, an RNAse III family nuclease, and function through RNA-mediated interference (RNAi) and related pathways to regulate the expression of target genes (Hannon, 2002, Nature 418:244-251; Pasquinelli, et al., 2002, Annu. Rev. Cell. Dev. Biol. 18:495-513, all of which are herein incorporated by reference). miRNAs may be configured to permit experimental manipulation of gene expression in mammalian cells as synthetic silencing triggers ‘short hairpin RNAs’ (shRNAs) (Paddison, et al., 2002, Cancer Cell 2:17-23, herein incorporated by reference). Silencing by shRNAs involves the RNAi machinery and correlates with the production of small interfering RNAs (siRNAs), which are a signature of RNAi.

The term “target RNA molecule” refers to an RNA molecule to which an RNAi molecule is homologous or complementary. Typically, when such homology or complementary is about 100%, the RNAi is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of RNAi. Such targets include unprocessed mRNA, ribosomal RNA, and viral RNA genomes.

The term “RNA function” refers to the role of an RNA molecule in a cell. For example, the function of mRNA is translation into a protein. Other RNAs are not translated into a protein, and have other functions; such RNAs include but are not limited to transfer RNA (tRNA), ribosomal RNA (rRNA), and small nuclear RNAs (snRNAs). An RNA molecule may have more than one role in a cell.

The term “inhibition” when used in reference to gene expression or RNA function refers to a decrease in the level of gene expression or RNA function as the result of some interference with or interaction with gene expression or RNA function as compared to the level of expression or function in the absence of the interference or interaction. The inhibition may be complete, in which there is no detectable expression or function, or it may be partial. Partial inhibition can range from near complete inhibition to near absence of inhibition; typically, inhibition is at least about 50% inhibition, or at least about 80% inhibition, or at least about 90% inhibition.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and, where the RNA encodes a protein, into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “vector” refers to nucleic acid molecules that transfer DNA segment(s) from one cell to another, and includes those nucleic acid molecules that are viral in origin. The term “vehicle” is sometimes used interchangeably with “vector.” A vector may be used to transfer an expression cassette into a cell; in addition or alternatively, a vector may comprise additional genes, including but not limited to genes which encode marker proteins, by which cell transfection can be determined, selection proteins, be means of which transfected cells may be selected from non-transfected cells, or reporter proteins, by means of which an effect on expression or activity or function of the reporter protein can be monitored.

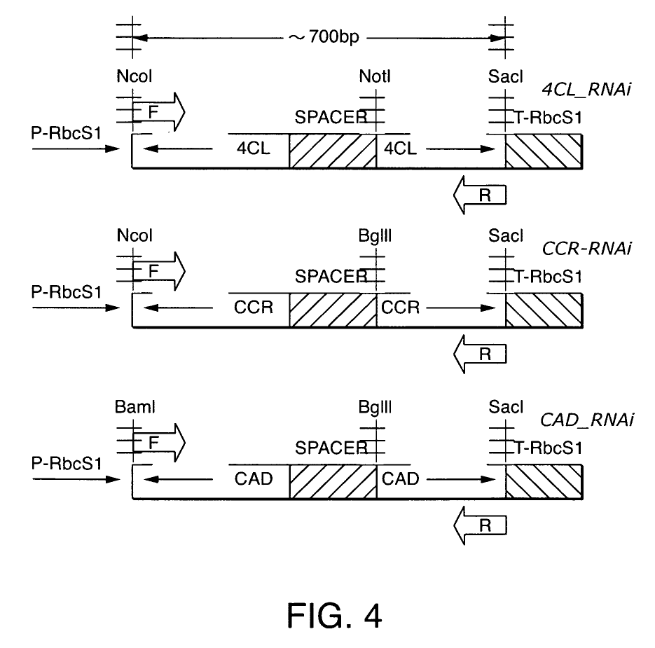

The term “expression vector” refers to a vector comprising one or more expression cassettes. Such expression cassettes include those of the present invention, where expression results in an RNAi transcript, such as expression cassettes shown in FIG. 4. As used herein, an expression vector is capable of expressing a silencing construct for altering lignin, such as an ImpactVector™ 1.1.

The term “expression cassette” refers to a chemically synthesized or recombinant DNA molecule containing a desired coding sequence and appropriate nucleic acid sequences necessary for the expression of the operably linked coding sequence either in vitro or in vivo. Expression in vitro includes expression in transcription systems and in transcription/translation systems. Expression in vivo includes expression in a particular host cell and/or organism. Nucleic acid sequences necessary for expression in prokaryotic cell or in vitro expression system usually include a promoter, an operator (optional), and a ribosome binding site, often along with other sequences. Eukaryotic in vitro transcription systems and cells are known to utilize promoters, enhancers, and termination and polyadenylation signals. Nucleic acid sequences useful for expression via bacterial RNA polymerases, referred to as a transcription template in the art, include a template DNA strand which has a polymerase promoter region followed by the complement of the RNA sequence desired. In order to create a transcription template, a complementary strand is annealed to the promoter portion of the template strand. However, the present invention is not limited to any particular configuration and all known systems are contemplated.

The term “transgene” as used herein refers to a foreign gene that is placed into an organism by introducing the foreign gene into a cell. The term “foreign gene” refers to any nucleic acid (e.g., antisense sequence or gene sequence) that is introduced into the genome of an animal by experimental manipulations and may include nucleotide sequences found in that plant so long as the introduced nucleotide or gene does not reside in the same location as does the naturally-occurring gene.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, bacterial infection, viral infection, biolistics (i.e., particle bombardment) and the like.

The terms “transfect” and “transform” (and grammatical equivalents, such as “transfected” and “transformed”) are used interchangeably herein.

The term “stable transfection” or “stably transfected” refers to the introduction and integration of foreign DNA into the genome of the transfected cell.

The term “stable transfectant” refers to a cell which has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes.

The term “transient transfectant” refers to cells that have taken up foreign DNA but have failed to integrate this DNA.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are incorporated herein by reference), and are commercially available (e.g., the helium gas-driven microprojectile accelerator (PDS-1000/He, BioRad).

The term “host cell” refers to any cell capable of replicating and/or transcribing and/or translating a heterologous gene. Thus, a “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the codA gene is commonly used as a negative selectable marker in plants. Expression of the codA gene in cells grown in the presence of 5-fluorocytosine (5-FC) is cytotoxic; thus, growth of cells in selective medium containing g 5-fluorocytosine (5-FC) selects against cells capable of expressing a functional codA enzyme.

The term “reporter gene” refers to a gene encoding a protein that may be assayed. Examples of reporter genes include, but are not limited to, B-glucuronidase (GUS) (pBI221 (Clontech—Catalog#6019-1, Palo Alto, Calif. vector) or luciferase (See, e.g., deWet et al., Mol. Cell. Biol. 7:725 (1987) and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif.), chloramphenicol acetyltransferase, β-galactosidase, alkaline phosphatase, and horse radish peroxidase.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is that which is most frequently observed in a population and is thus arbitrarily designated the “normal” or “wild-type” form of the gene.

In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “modify” in reference to lignin refers to altering a lignin structure, for example by silencing a gene in the lignin biosynthesis pathway. An altered lignin structure may be any one of the numerous types of structures associated with lignin.

The terms “in operable combination,” “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987, herein incorporated by reference). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987, herein incorporated by reference).

The terms “promoter element,” “promoter,” or “promoter sequence” as used herein, refer to a DNA sequence that is located at the 5′ end (i.e. precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of RNA from the gene. The promoter, therefore, serves as a transcriptional regulatory element and also provides a site for initiation of transcription of the gene into RNA.

Promoters may be tissue specific or cell specific. The term “tissue specific” as it applies to a promoter refers to a promoter that is capable of directing selective expression of a nucleotide sequence of interest to a specific type of tissue in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York (1989) pp. 16.7-16.8, herein incorporated by reference). A commonly used splice donor and acceptor site is the splice junction from the 16S RNA of SV40.

The term “purified” refers to molecules, either nucleic acid or amino acid sequences that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “sample” is used in its broadest sense. In one sense it can refer to a plant cell or tissue. In another sense, it is meant to include a specimen or culture obtained from any source, as well as biological and environmental samples. Biological samples may be obtained from plants or animals and encompass seeds, kernels, pollen, leaves, stalks, whole plants, feedstock biomass, fluids, solids, tissues, and gases. Environmental samples include environmental material such as surface matter, soil, water, and industrial samples. These examples are not to be construed as limiting the sample types applicable to the present invention.

The term “bar” refers to a phosphinothricin acetyl transferase gene (Thompson et al., EMBO J. 6:2519 2523 (1987), herein incorporated by reference). The bar gene is a selectable marker for herbicide resistance. The 5′ end of bar is operably linked to the rice actin 1 gene promoter which has been shown to operable in maize (Zhong et al., Plant Physiology 110: 1097 1107 (1996); Zhang et al., Theor. Appl. Genet. 92: 752 761 (1996); Zhang et al., Plant Science 116: 73 84 (1996), all of which are herein incorporated by reference). The 3′ end of bar is operably linked to the nos 3′ untranslated sequences.

The term “cytoplasm” as used herein refers to the organized complex of inorganic and organic substances external to the nuclear membrane of a cell and including the cytosol and membrane-bound organelles (i.e., for example, mitochondria or chloroplasts).

### DETAILED DESCRIPTION OF THE INVENTION

The present invention relates to gene-specific silencing of genes involved in lignin biosynthesis through RNA interference or other methods, and in particular, to vectors for expressing RNAi molecules that inhibit the expression of genes in the lignin biosynthesis pathway. In some embodiments, the present invention provides compositions and methods for inducible or constitutive expression of RNAi molecules, and/or for long-term expression of RNAi molecules. Hence the compositions and methods described herein are suitable for regulatable and/or sustained gene-specific silencing in cells, and further for silencing genes altering lignin and/or cellulose biosynthesis.

RNA interference (RNAi) is a post-transcriptional gene silencing process that is induced by a dsRNA (a small interfering RNA; siRNA), and has been used to modulate gene expression. Generally, RNAi has been performed by contacting cells with a double stranded siRNA. However, manipulation of RNA outside of cell is tedious due to the sensitivity of RNA to degradation.

It is contemplated that biomass conversion costs will be reduced by developing crop varieties comprising altered lignin content. In some embodiments, the present invention provides crops that endogenously produce cellulase enzymes for enhancing cellulose degradation and/or ligninase enzymes for enhancing subsequent lignin degradation, or plants that have increased cellulose or an overall biomass yield applicable to the desired product, (Curr Opin Biotechnol. 2006 June; 17(3):315-9. Epub 2006 May 15; herein incorporated by reference). In some embodiments, the RNAi constructs are introduced into corn plants described in U.S. Pat. No. 7,049,485; herein incorporated by reference in its entirety.

In particular, the present invention provides methods for reducing corn biofuel production costs. In some embodiments, the present invention provides methods and gene constructs for downregulating one or more of the lignin biosynthesis enzymes presented in the pathway depicted in FIG. 1. In preferred embodiments, corn-specific gene constructs encoding RNAi's specific for one or more of the above enzymes are introduced into corn lines, preferably elite corn lines. In some preferred embodiments, lignin content of leaves, stems and roots of each down regulated plant compared with control non-transgenic plants of the same age using near infrared spectrophotometry.

Among four different corn bm mutants, lignin content was reduced between 8% and 30% based on the location of the mutated enzyme in the lignin biosynthesis pathway (Chabbert, et al., 1994, J. Sci. Food. Agric. 64:349-355, herein incorporated by reference). Studies on down-regulation of lignin or modification of lignin structure have been reported in several crops, except in corn, via down regulation of different enzymes involved with lignin biosynthesis pathway (Sticklen, 2006, Current Opin. Biotech. 17(3):315-319, herein incorporated by reference). Down regulation of 4CL (FIG. 1) in transgenic quaking aspen (Populus tremuloides) resulted in a 45% decrease in lignin with a concomitant 15% increase in cellulose, doubling the plant cellulose to lignin ratio without any change in lignin composition and without any apparent harm to plant growth, development and structural integrity (Hu, et al., 1999, Nature Biotech. 17:808-812, herein incorporated by reference). Such compensation showed that the quantitative or qualitative changes of one cell wall component often results in alteration of other call wall components (Boudet, et al., 2003, Trends Plant Sci. 8:576-581, herein incorporated by reference). Thus by decreasing lignin biosynthesis can reduce the costs of pretreatment processes and/or increasing cellulose biosythesis would increase the level of fermentable sugars from corn biomass.

Brown-midrib (bm) mutants of forages have aroused considerable agronomic interest over the past 30 years due to their reduced lignin content and corresponding improved digestibility (for a review see Chemey et al. 1991, Adv. Agron. 46:157-198, herein incorporated by reference). The mutants are phenotypically characterised by the presence of a reddish-brown pigment in leaf midribs and stem sclerenchyma. While the identity of the chromophore is not known, the pigment has been shown to be closely associated with lignin, persisting in the cell wall residue after cellulose and hemicellulose have been removed. Four distinct naturally occurring mutants have been described for maize (bm1, bm2, bm3 and bm4), while chemically induced mutants exist in sorghum and pearl millet.

Although the relationship between lignin and forage digestibility has long been appreciated, it is not yet fully understood. It is likely that both the total lignin content and the lignin monomer composition have an effect. Lignin is a heterogeneous aromatic polymer which, in grasses, is composed of the monolignols para-coumaryl, coniferyl and sinapyl alcohol. The structure is complex, incorporating ether and carbon-carbon linkages between monomers with extensive cross-links, probably via hydroxycinnamic acid bridges, to other cell wall polymers. The content of hydroxycinnamic acids, especially p-coumaric and ferulic acid, has been inversely correlated with cell wall digestibility and is altered in some brown-midrib mutants (Kuc and Nelson 1964, Arch. Biochem. Biophys. 105:103; Kuc et al. 1968, Phytochemistry 7:1435-1436; all of which are herein incorporated by reference).

Monolignols and hydroxycinnamic acids are products of the phenylpropanoid pathway which also supplies intermediates for the synthesis of phytoalexins, flavonoids and tannins (Whetten and Sederoff 1995; herein incorporated by reference). Of the many enzymes on this pathway only cinnamoyl CoA-reductase (CCR) and cinnamyl alcohol dehydrogenase (CAD) are dedicated solely to monolignol synthesis. In addition, lignin-specific O-methyltransferases (OMT) have been reported in a number of species (e.g. Bugos et al., 1991, Plant Mol. Biol. 17:203, herein incorporated by reference). This enzyme catalyses the conversion of para-coumaric acid, via the intermediates caffeic acid and 5-hydroxyferulic acid, to the methoxylated derivatives ferulic and sinapic acid. Para-coumaric acid, ferulic acid and sinapic acid are ultimately converted, via cinnamyl CoA ligase, CCR and CAD, to the monolignols p-coumaric, coniferyl and syringyl alcohol which give rise to p-hydroxyphenyl, guaiacyl and syringyl lignins.

Although the genomic location of maize bm mutations has been known for over 60 years (Jorgensen, 1931, J Am Soc Agron. 23:549-557, herein incorporated by reference), thus far only one of the mutant genes has been identified (Vignols et al. 1995, Plant Cell, 7:407-416, herein incorporated by reference). Maize bm3 is severely deficient in catechol-OMT activity, with only 10% of the activity found in normal plants (Grand et al. 1985, Physiol. Veg., 23:905-911; herein incorporated by reference). Recent work has confirmed that the OMT gene is indeed the site of the bm3 mutation (Vignols et al. 1995, Plant Cell, 7:407-416, herein incorporated by reference). Biochemical evidence suggests that other brown-midrib plants may also be deficient in lignin biosynthetic enzymes. Our own unpublished work shows that OMT activity is reduced in sorghum bmr12 and bmr18 while both OMT and CAD activities are reduced in sorghum bmr6 (Bucholtz et al. 1980, Agric. Food Chem., 28:1239-1241; Pillonel et al. 1991, Planta, 185, 538 544; all of which are herein incorporated by reference). In particular, Maize bm1 has reduced CAD enzyme, bm1 plants have altered lignin.

Numerous attempts were made in order to reduce lignin in plants using genetic engineering of the lignin biochemical pathway. Most notably increasing FSH production under control of a Ca35S promoter in Arabidopsis plants (U.S. Pat. No. 6,489,538), Genes encoding seven enzymes of the monolignol pathway were independently down-regulated in alfalfa (Medicago sativa) using antisense and RNA interference, under control of a pal2, bean phenylalanine-ammonia-lyase 2 promoter, reducing total flux into lignin (Chen, et al., The Plant Journal (2006) 48, 113-124), and Reddy, et al., PNAS, 2005, 102 (46): 16573-16578, herein incorporated by reference). Further, transgenic corn lines were developed for reducing lignin postharvest to more readily allow access of hydrolyzing enzymes to the cellulose material, with the expression of lignin digesting enzymes in plastids that were released upon mashing of the maize stover (U.S. Pat. No. 7,049,485, herein incorporated by reference).

Sustainable agriculture methods and production of alcohols for use as alternative fuels support the use of lignocellulosic biomass. However, the lignin component is the rate limiting step and means of reducing lignin are contemplated to provide more economical ways of producing alcohols (Sticklen, Crop Sci. 47:2238-2248 (2007)). Although several methods were reported for reducing lignin in plants, including maize plants, these are not yet economically desirable (Sticklen, Crop Sci. 47:2238-2248 (2007)). However, lignocellulosic biomass processing should be a more sustainable process to provide alcohol based fuels (Sticklen, Crop Sci. 47:2238-2248 (2007), herein incorporated in its entirety). Therefore, there remains a need for maize plants with reduced lignin content.

The following are exemplary genes contemplated for use in the present inventions for altering lignin structures, in particular for silencing lignin altering genes by an RNAi vector of the present inventions: Cinnamoyl CoA:NADP oxidoreductase (CCR, EC 1.2.1.44) catalyzes the conversion of cinnamoyl CoA esters to their corresponding cinnamaldehydes; aldehyde O-methyltransferase gene, AldOMT; Caffeic acid 3-O-methyltransferase (CAOMT) and caffeoyl-coenzyme A 3-O-methyltransferase (CCoAOMT); Phenylalanine ammonia-lyase or PAL refers to the first enzyme of the phenylpropanoid pathway and catalyzes the deamination of phenylalanine to produce trans-cinnamic acid. Twelve clusters with high similarities to PAL genes were found; Cinnamate 4-hydroxylase or C4H belongs to the CYP73A group of cytochrome P450-dependent monooxygenases protein family for hydroxylates cinnamic acid to generate p-coumaric acid; 4-Hydroxycinnamoyl CoA ligase or 4CL is responsible for the CoA esterification of p-coumaric acid, caffeic acid, ferulic acid, 5-hydroxyferulic acid, and sinapic acid, Eucalyptus, 3 clusters encoding 4CLs were found; Hydroxycinnamoyl-CoA:shikimate/quinate hydroxycinnamoyltransferase (HCT) was recently purified and the corresponding gene cloned from tobacco (Hoffmann et al., 2003, J. Biol. Chem. 278, 95-103; herein incorporated by reference), converts p-coumaroyl-CoA and caffeoyl-CoA to their corresponding shikimate or quinate esters and catalyzes the reverse reaction as well, Shikimate and quinate esters of p-coumaroyl-CoA have been shown to be preferred substrates for p-coumarate 3-hydroxylase (C3H), which converts them into their corresponding caffeoyl esters (Schoch et al., 2001, J. Biol. Chem. 276, 36566-36574; herein incorporated by reference); p-Coumarate 3-hydroxylase or C3H belongs to the CYP98A3 group of cytochrome P450-dependent monooxygenases family. Although its name indicates p-coumaric acid as substrate, as commented before, the shikimate and quinate esters of this acid are the substrates instead (Schoch et al., 2001, J. Biol. Chem. 276, 36566-36574; herein incorporated by reference); Caffeoyl CoA O-methyltransferase or CCoAOMT catalyzes methylation of caffeoyl CoA to generate feruloyl CoA. Four clusters encoding CCoAOMT proteins were found in Eucalyptus; Cinnamoyl CoA reductase or CCR converts hydroxycinnamoyl CoA esters to their corresponding aldehydes; Ferulate 5-hydroxylase or F5H or coniferaldehyde 5-hydroxylase (CAld5H) refers to a cytochrome P450-dependent monooxygenase of the CYP84 group that converts ferulic acid to 5-hydroxyferulic acid and under certain conditions, preferentially converts coniferaldehyde and/or coniferyl alcohol to synapaldehyde and/or sinapyl alcohol, respectively (Humphreys et al., 1999, Proc Natl Acad Sci USA 96:10045-10050; Osakabe et al., 1999, Proc. Natl. Acad. Sci. USA 96:8955-8960; all of which are herein incorporated by reference); Caffeic acid O-methyltransferase or COMT was shown by in vitro studies using recombinant alfalfa (Medicago sativa) and sweetgum COMTs that preferential substrates are 5-hydroxyconiferaldehyde and/or 5-hydroxyconiferyl alcohol, resulting in sinapaldehyde and/or sinapyl alcohol, respectively (Osakabe et al., 1999, Proc. Natl. Acad. Sci. USA 96:8955-8960; Parvathi et al., 2001, The Plant Journal, 2001, 25(2): 193-202; all of which are herein incorporated by reference); Cinnamyl alcohol dehydrogenase or CAD catalyze the conversion of cinnamyl aldehydes into their corresponding alcohols. Plants show a large variety of CADs that reduce a wide range of aldehydes, many of which are expressed in response to pathogen infection (Walter et al., 1988, Proc Natl Acad Sci USA 85 5546-5550; herein incorporated by reference); and enzymes related to lignin biosynthesis and wood formation; Chitinase substrates or products of the class I chitinases-mediated reaction (Zhong et al., 2002, The Plant Cell, Vol. 14, 165-179; herein incorporated by reference); Laccase—an promote polymerization of monolignols in the absence of H₂O₂, resulting in either lignans or lignins, extracellular localization is in accordance to the proposed role of laccases as polymerization catalysts of monolignols. Interestingly, cluster EGEZRT3005B09.g encodes a laccase protein 67.2% identical to that encoded by the poplar lac3 gene, which, when silenced, causes alterations in phenolics metabolism and cell wall structure (Ranocha et al., 2002; herein incorporated by reference); and Dirigent protein—promote stereoselective coupling of monolignols and their role in the formation of (+)-pinoresinol lignan in Forsythia sp and western red cedar (Thuja plicata) has been well established (Davin et al., 1997, Science 275: 362-366; Gang et al., 1999, Chem Biol 6: 143-151; Kim, et al., 2002, Phytochemistry, (2002), 61:311-322; all of which are herein incorporated by reference). However, as noted in FIG. 1B, recent information has modified how many of these enzymes alter lignin, and point out that these enzymes are providing molecules and structures that are plant specific, in other words the homologes of these enzymes are not providing the same lignin molecules in other plants.

TABLE 1
Exemplary sequences for RNA targets.

| Zea mays | mRNA SEQ ID NO: | Protein SEQ ID NO: |
|---|---|---|
| cinnamoyl CoA | 1 | 7 |
| reductase | | |
| 4-coumarate coenzyme | 2 | 8 |
| A ligase (4CL) | | |
| cinnamyl alcohol | 3 | 9 |
| dehydrogense (CAD) | | |
| cinnamoyl CoA | 4 | 10 |
| reductase | | |
| caffeoyl-CoA 3-O- | 5 | 11 |
| methyltransferase 1 | | |
| (ccoaomt1) | | |
| caffeoyl CoA 3-O- | 6 | 12 |
| methyltransferase | | |
| (ccoaomt2) | | |

RNAi refers to the introduction of homologous double stranded RNA (dsRNA) to target a specific gene product, resulting in post-transcriptional silencing of that gene. This phenomena was first reported in Caenorhabditis elegans by Guo and Kemphues (Par-1, A gene required for establishing polarity in C. elegans embryos, encodes a putative Ser/Thr kinase that is asymmetrically distributed, 1995, Cell, 81(4) 611-620; herein incorporated by reference) and subsequently Fire et al. (Potent and specific genetic interference by double-stranded RNA in Caenorhabditis elegans, 1998, Nature 391:806-811; herein incorporated by reference) discovered that it is the presence of dsRNA, formed from the annealing of sense and antisense strands present in the in vitro RNA preps, that is responsible for producing the interfering activity.

The present invention contemplates the use of RNA interference (RNAi) to downregulate the expression of genes needed for lignin biosynthesis, thus reducing the cost of producing biofeuls. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs.

Carthew has reported (Curr. Opin. Cell Biol. 13(2):244-248 (2001); herein incorporated by reference) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

In preferred embodiments, the dsRNA used to initiate RNAi, may be isolated from native source or produced by known means, e.g., transcribed from DNA. The promoters and vectors described in more detail below are suitable for producing dsRNA. RNA is synthesized either in vivo or in vitro. In some embodiments, endogenous RNA polymerase of the cell may mediate transcription in vivo, or cloned RNA polymerase can be used for transcription in vivo or in vitro. In other embodiments, the RNA is provided transcription from a transgene in vivo or an expression construct. In some embodiments, the RNA strands are polyadenylated; in other embodiments, the RNA strands are capable of being translated into a polypeptide by a cell's translational apparatus. In still other embodiments, the RNA is chemically or enzymatically synthesized by manual or automated reactions. In further embodiments, the RNA is synthesized by a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. In some embodiments, the RNA is dried for storage or dissolved in an aqueous solution. In other embodiments, the solution contains buffers or salts to promote annealing, and/or stabilization of the duplex strands.

In some embodiments, the dsRNA is transcribed from the vectors as two separate stands. In other embodiments, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. In some embodiments, a DNA duplex provided at each end with a promoter sequence can directly generate RNAs of defined length, and which can join in pairs to form a dsRNA. See, e.g., U.S. Pat. No. 5,795,715, incorporated herein by reference. RNA duplex formation may be initiated either inside or outside the cell.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases.

There is no upper limit on the length of the dsRNA that can be used. For example, the dsRNA can range from about 21 base pairs (bp) of the gene to the full length of the gene or more. In one embodiment, the dsRNA used in the methods of the present invention is about 1000 bp in length. In another embodiment, the dsRNA is about 500 bp in length. In yet another embodiment, the dsRNA is about 22 bp in length. In some preferred embodiments, the sequences that mediate RNAi are from about 21 to about 23 nucleotides. That is, the isolated RNAs of the present invention mediate degradation of the target RNA (e.g., the RNA encoding one or more of the lignin biosynthesis pathway genes described above).

The double stranded RNA of the present invention need only be sufficiently similar to natural RNA that it has the ability to mediate RNAi for the target RNA. In one embodiment, the present invention relates to RNA molecules of varying lengths that direct cleavage of specific mRNA to which their sequence corresponds. It is not necessary that there be perfect correspondence of the sequences, but the correspondence must be sufficient to enable the RNA to direct RNAi cleavage of the target mRNA. In a particular embodiment, the RNA molecules of the present invention comprise a 3′ hydroxyl group. In some embodiments, the amount of target RNA (mRNA) is reduced in the cells of the target organism (e.g., H. glycines) exposed to target specific double stranded RNA as compared to target organisms that have not been exposed to target specific double stranded RNA.

Accordingly, in some embodiments, the present invention provides isolated RNA molecules (double-stranded or single-stranded) that are complementary to sequences required for lignin biosynthesis. In some embodiments, the RNA molecules utilized mediate RNAi for one or more of the lignin biosynthesis enzymes identified in FIG. 1.

In some embodiments, probes that are specific for a lignin biosynthesis pathway gene of interest are amplified from a DNA sample prepared from maize by using primers designed from maize genomic DNA or cDNA. Genes amplified from maize DNA are then used as probes for homologous genes from a genomic or cDNA libraries prepared from a maize line of interest. These genes are then inserted into an expression vector so that a nematode double stranded RNA corresponding to the gene of interest is produced when the vector is used to transfect a plant.

In some embodiments, the present invention provides transgenic plants that express dsRNA molecules that correspond to target lignin biosynthesis pathway molecules. A heterologous gene encoding a RNAi gene of the present invention, which includes variants of the RNAi gene, includes any suitable sequence that encodes an double stranded molecule specific for a lignin biosynthesis pathway target RNA. Preferably, the heterologous gene is provided within an expression vector such that transformation with the vector results in expression of the double stranded RNA molecule; suitable vectors are described below.

In yet other embodiments of the present invention, a transgenic plant comprises a heterologous gene encoding a RNAi gene of the present invention operably linked to an inducible promoter, and is grown either in the presence of the an inducing agent, or is grown and then exposed to an inducing agent. In still other embodiments of the present invention, a transgenic plant comprises a heterologous gene encoding a RNAi gene of the present invention operably linked to a promoter which is either tissue specific or developmentally specific, and is grown to the point at which the tissue is developed or the developmental stage at which the developmentally-specific promoter is activated. Such promoters include seed and root specific promoters. In still other embodiments of the present invention, the transgenic plant comprises a RNAi gene of the present invention operably linked to constitutive promoter. In further embodiments, the transgenic plants of the present invention express at least one double stranded RNA molecule at a level sufficient to reduce the proliferation of nematodes as compared to the proliferation of nematodes observed in a nontransgenic plant.

The methods of the present invention are not limited to any particular plant. Indeed, a variety of plants are contemplated, including but not limited to soybean, wheat, oats, milo, sorghum, cotton, tomato, potato, tobacco, pepper, rice, maize, barley, Brassica, Arabidopsis, sunflower, poplar, pineapple, banana, turf grass, poplar and pine. Many commercial cultivars can be transformed with heterologous genes. In cases where that is not possible, non-commercial cultivars of plants can be transformed, and the trait for expression of the RNAi gene of the present invention moved to commercial cultivars by breeding techniques well-known in the art. In some preferred embodiments, transgenic maize plants are produced as described in U.S. Pat. No. 7,049,485; herein incorporated by reference in its entirety.

The methods of the present invention contemplate the use of at least one heterologous gene encoding a RNAi gene of the present invention. Heterologous genes intended for expression in plants are first assembled in expression cassettes comprising a promoter. Methods which are well known to those skilled in the art may be used to construct expression vectors containing a heterologous gene and appropriate transcriptional and translational control elements. These methods include in vitro recombinant DNA techniques, synthetic techniques, and in vivo genetic recombination. Such techniques are widely described in the art (See e.g., Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.; herein incorporated by reference).

In general, these vectors comprise a nucleic acid sequence of the invention encoding a RNAi gene of the present invention (as described above) operably linked to a promoter and other regulatory sequences (e.g., enhancers, polyadenylation signals, etc.) required for expression in a plant.

Promoters include but are not limited to constitutive promoters, tissue-, organ-, and developmentally-specific promoters, and inducible promoters. Examples of promoters include but are not limited to: constitutive promoter 35S of cauliflower mosaic virus; a wound-inducible promoter from tomato, leucine amino peptidase (“LAP,” Chao et al. (1999) Plant Physiol 120: 979-992; herein incorporated by reference); a chemically-inducible promoter from tobacco, Pathogenesis-Related 1 (PR1) (induced by salicylic acid and BTH (benzothiadiazole-7-carbothioic acid S-methyl ester)); a tomato proteinase inhibitor II promoter (PIN2) or LAP promoter (both inducible with methyl jasmonate); a heat shock promoter (U.S. Pat. No. 5,187,267; herein incorporated by reference); a tetracycline-inducible promoter (U.S. Pat. No. 5,057,422; herein incorporated by reference); and seed-specific promoters, such as those for seed storage proteins (e.g., phaseolin, napin, oleosin, and a promoter for soybean beta conglycin (Beachy et al. (1985) EMBO J. 4: 3047-3053); herein incorporated by reference). In some preferred embodiments, the promoter is a phaseolin promoter.

The expression cassettes may further comprise any sequences required for expression of mRNA. Such sequences include, but are not limited to transcription terminators, enhancers such as introns, viral sequences, and sequences intended for the targeting of the gene product to specific organelles and cell compartments.

A variety of transcriptional terminators are available for use in expression of sequences using the promoters of the present invention. Transcriptional terminators are responsible for the termination of transcription beyond the transcript and its correct polyadenylation. Appropriate transcriptional terminators and those which are known to function in plants include, but are not limited to, the CaMV 35S terminator, the tml terminator, the pea rbcS E9 terminator, and the nopaline and octopine synthase terminator (See e.g., Odell et al. (1985) Nature 313:810; Rosenberg et al. (1987) Gene, 56:125; Guerineau et al. (1991) Mol. Gen. Genet., 262:141; Proudfoot (1991) Cell, 64:671; Sanfacon et al. Genes Dev., 5:141; Mogen et al. (1990) Plant Cell, 2:1261; Munroe et al. (1990) Gene, 91:151; Ballad et al. (1989) Nucleic Acids Res. 17:7891; Joshi et al. (1987) Nucleic Acid Res., 15:9627; all of which are herein incorporated by reference).

In addition, in some embodiments, constructs for expression of the gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. For example, the introns of the maize Adh1 gene have been found to significantly enhance the expression of the wild-type gene under its cognate promoter when introduced into maize cells (Calais et al. (1987) Genes Develop. 1:1183; herein incorporated by reference). Intron sequences have been routinely incorporated into plant transformation vectors, typically within the non-translated leader.

In some embodiments of the present invention, the construct for expression of the nucleic acid sequence of interest also includes a regulator such as a nuclear localization signal (Calderone et al. (1984) Cell 39:499; Lassoer et al. (1991) Plant Molecular Biology 17:229; all of which are herein incorporated by reference), a plant translational consensus sequence (Joshi (1987) Nucleic Acids Research 15:6643; herein incorporated by reference), an intron (Luehrsen and Walbot (1991) Mol. Gen. Genet. 225:81; herein incorporated by reference), and the like, operably linked to the nucleic acid sequence encoding ADS.

In preparing a construct comprising a nucleic acid sequence encoding a RNAi gene of the present invention, various DNA fragments can be manipulated, so as to provide for the DNA sequences in the desired orientation (e.g., sense or antisense) orientation. For example, adapters or linkers can be employed to join the DNA fragments or other manipulations can be used to provide for convenient restriction sites, removal of superfluous DNA, removal of restriction sites, or the like. For this purpose, in vitro mutagenesis, primer repair, restriction, annealing, resection, ligation, or the like is preferably employed, where insertions, deletions or substitutions (e.g., transitions and transversions) are involved.

Numerous transformation vectors are available for plant transformation. The selection of a vector for use will depend upon the preferred transformation technique and the target species for transformation. For certain target species, different antibiotic or herbicide selection markers are preferred. Selection markers used routinely in transformation include the nptII gene which confers resistance to kanamycin and related antibiotics (Messing and Vierra (1982) Gene 19: 259; Bevan et al. (1983) Nature 304:184; all of which are herein incorporated by reference), the bar gene which confers resistance to the herbicide phosphinothricin (White et al. (1990) Nucl Acids Res. 18:1062; Spencer et al. (1990) Theor. Appl. Genet. 79:625; all of which are herein incorporated by reference), the hph gene which confers resistance to the antibiotic hygromycin (Blochlinger and Diggelmann (1984) Mol. Cell. Biol. 4:2929; herein incorporated by reference), and the dhfr gene, which confers resistance to methotrexate (Bourouis et al. (1983) EMBO J., 2:1099; herein incorporated by reference).

In some preferred embodiments, the vector is adapted for use in an Agrobacterium mediated transfection process (See e.g., U.S. Pat. Nos. 5,981,839; 6,051,757; 5,981,840; 5,824,877; and 4,940,838; all of which are incorporated herein by reference). Construction of recombinant Ti and Ri plasmids in general follows methods typically used with the more common bacterial vectors, such as pBR322. Additional use can be made of accessory genetic elements sometimes found with the native plasmids and sometimes constructed from foreign sequences. These may include but are not limited to structural genes for antibiotic resistance as selection genes.

There are two systems of recombinant Ti and Ri plasmid vector systems now in use. The first system is called the “cointegrate” system. In this system, the shuttle vector containing the gene of interest is inserted by genetic recombination into a non-oncogenic Ti plasmid that contains both the cis-acting and trans-acting elements required for plant transformation as, for example, in the pMLJ1 shuttle vector and the non-oncogenic Ti plasmid pGV3850. The second system is called the “binary” system in which two plasmids are used; the gene of interest is inserted into a shuttle vector containing the cis-acting elements required for plant transformation. The other necessary functions are provided in trans by the non-oncogenic Ti plasmid as exemplified by the pBIN19 shuttle vector and the non-oncogenic Ti plasmid PAL4404. Some of these vectors are commercially available.

In other embodiments of the invention, the nucleic acid sequence of interest is targeted to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences, as previously described (U.S. Pat. No. 5,501,967; herein incorporated by reference). One of skill in the art knows that homologous recombination may be achieved using targeting vectors which contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

In yet other embodiments, the nucleic acids of the present invention are utilized to construct vectors derived from plant (+) RNA viruses (e.g., brome mosaic virus, tobacco mosaic virus, alfalfa mosaic virus, cucumber mosaic virus, tomato mosaic virus, and combinations and hybrids thereof). Generally, the inserted ADS polynucleotide of the present invention can be expressed from these vectors as a fusion protein (e.g., coat protein fusion protein) or from its own subgenomic promoter or other promoter. Methods for the construction and use of such viruses are described in U.S. Pat. Nos. 5,846,795; 5,500,360; 5,173,410; 5,965,794; 5,977,438; and 5,866,785, all of which are incorporated herein by reference.

In some embodiments of the present invention the nucleic acid sequence of interest is introduced directly into a plant. One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator (WO 93/07278; herein incorporated by reference).

Once a nucleic acid sequence encoding an RNAi of the present invention is operatively linked to an appropriate promoter and inserted into a suitable vector for the particular transformation technique utilized (e.g., one of the vectors described above), the recombinant DNA described above can be introduced into the plant cell in a number of art-recognized ways. Those skilled in the art will appreciate that the choice of method might depend on the type of plant targeted for transformation. In some embodiments, the vector is maintained episomally. In other embodiments, the vector is integrated into the genome.

In some embodiments, the vector is introduced through ballistic particle acceleration using devices (e.g., available from Agracetus, Inc., Madison, Wis. and Dupont, Inc., Wilmington, Del.). (See e.g., U.S. Pat. No. 4,945,050; and McCabe et al. (1988) Biotechnology 6:923). See also, Weissinger et al. (1988) Annual Rev. Genet. 22:421; Sanford et al. (1987) Particulate Science and Technology, 5:27 (onion); Svab et al. (1990) Proc. Natl. Acad. Sci. USA, 87:8526 (tobacco chloroplast); Christou et al. (1988) Plant Physiol., 87:671 (soybean); McCabe et al. (1988) Bio/Technology 6:923 (soybean); Klein et al. (1988) Proc. Natl. Acad. Sci. USA, 85:4305 (maize); Klein et al. (1988) Bio/Technology, 6:559 (maize); Klein et al. (1988) Plant Physiol., 91:4404 (maize); Fromm et al. (1990) Bio/Technology, 8:833; and Gordon-Kamm et al. (1990) Plant Cell, 2:603 (maize); Koziel et al. (1993) Biotechnology, 11:194 (maize); Hill et al. (1995) Euphytica, 85:119 and Koziel et al. (1996) Annals of the New York Academy of Sciences 792:164; Shimamoto et al. (1989) Nature 338: 274 (rice); Christou et al. (1991) Biotechnology, 9:957 (rice); Datta et al. (1990) Bio/Technology 8:736 (rice); European Patent Application EP 0 332 581 (orchardgrass and other Pooideae); Vasil et al. (1993) Biotechnology, 11: 1553 (wheat); Weeks et al. (1993) Plant Physiol., 102:1077 (wheat); Wan et al. (1994) Plant Physiol. 104: 37 (barley); Jahne et al. (1994) Theor. Appl. Genet. 89:525 (barley); Knudsen and Muller (1991) Planta, 185:330 (barley); Umbeck et al. (1987) Bio/Technology 5: 263 (cotton); Casas et al. (1993) Proc. Natl. Acad. Sci. USA 90:11212 (sorghum); Somers et al. (1992) Bio/Technology 10:1589 (oat); Torbert et al. (1995) Plant Cell Reports, 14:635 (oat); Weeks et al. (1993) Plant Physiol., 102:1077 (wheat); Chang et al., WO 94/13822 (wheat) and Nehra et al. (1994) The Plant Journal, 5:285 (wheat); all of which are herein incorporated by reference.

In other embodiments, direct transformation in the plastid genome is used to introduce the vector into the plant cell (See e.g., U.S. Pat. Nos. 5,451,513; 5,545,817; 5,545,818; PCT application WO 95/16783; all of which are herein incorporated by reference). The basic technique for chloroplast transformation involves introducing regions of cloned plastid DNA flanking a selectable marker together with the nucleic acid encoding the RNA sequences of interest into a suitable target tissue (e.g., using biolistics or protoplast transformation with calcium chloride or PEG). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al. (1990) PNAS, 87:8526; Staub and Maliga, (1992) Plant Cell, 4:39; all of which are herein incorporated by reference). The presence of cloning sites between these markers allowed creation of a plastid targeting vector introduction of foreign DNA molecules (Staub and Maliga (1993) EMBO J., 12:601; all of which are herein incorporated by reference). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga (1993) PNAS, 90:913; herein incorporated by reference). Other selectable markers useful for plastid transformation are known in the art and encompassed within the scope of the present invention. Plants homoplasmic for plastid genomes containing the two nucleic acid sequences separated by a promoter of the present invention are obtained, and are preferentially capable of high expression of the RNAs encoded by the DNA molecule.

In other embodiments, vectors useful in the practice of the present invention are microinjected directly into plant cells by use of micropipettes to mechanically transfer the recombinant DNA (Crossway (1985) Mol. Gen. Genet, 202:179; herein incorporated by reference). In still other embodiments, the vector is transferred into the plant cell by using polyethylene glycol (Krens et al. (1982) Nature, 296:72; Crossway et al. (1986) BioTechniques, 4:320; all of which are herein incorporated by reference); fusion of protoplasts with other entities, either minicells, cells, lysosomes or other fusible lipid-surfaced bodies (Fraley et al. (1982) Proc. Natl. Acad. Sci., USA, 79:1859; herein incorporated by reference); protoplast transformation (EP 0292435; herein incorporated by reference); direct gene transfer (Paszkowski et al. (1984) EMBO J., 3:2717; Hayashimoto et al. (1990) Plant Physiol. 93:857; all of which are herein incorporated by reference).

In still further embodiments, the vector may also be introduced into the plant cells by electroporation (Fromm, et al. (1985) Proc. Natl. Acad. Sci. USA 82:5824; Riggs et al. (1986) Proc. Natl. Acad. Sci. USA 83:5602; all of which are herein incorporated by reference). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In addition to direct transformation, in some embodiments, the vectors comprising a nucleic acid sequence encoding a RNAi gene of the present invention are transferred using Agrobacterium-mediated transformation (Hinchee et al. (1988) Biotechnology, 6:915; Ishida et al. (1996) Nature Biotechnology 14:745; herein incorporated by reference). Agrobacterium is a representative genus of the gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Heterologous genetic sequences (e.g., nucleic acid sequences operatively linked to a promoter of the present invention), can be introduced into appropriate plant cells, by means of the Ti plasmid of Agrobacterium tumefaciens. The Ti plasmid is transmitted to plant cells on infection by Agrobacterium tumefaciens, and is stably integrated into the plant genome (Schell (1987) Science, 237:1176; herein incorporated by reference). Species which are susceptible infection by Agrobacterium may be transformed in vitro. Alternatively, plants may be transformed in vivo, such as by transformation of a whole plant by Agrobacteria infiltration of adult plants, as in a “floral dip” method (BechtoldN, Ellis J, Pelletier G (1993) Cr. Acad. Sci. III—Vie 316:1194-1199; herein incorporated by reference).

After selecting for transformed plant material that can express the heterologous gene encoding a RNAi gene of the present invention, whole plants are regenerated. Plant regeneration from cultured protoplasts is described in Evans et al. (1983) Handbook of Plant Cell Cultures, Vol. 1: (MacMillan Publishing Co. New York); and Vasil I. R. (ed.), Cell Culture and Somatic Cell Genetics of Plants, Acad. Press, Orlando, Vol. I (1984), and Vol. III (1986); herein incorporated by reference. It is known that many plants can be regenerated from cultured cells or tissues, including but not limited to all major species of sugarcane, sugar beet, cotton, fruit and other trees, legumes and vegetables, and monocots (e.g., the plants described above). Means for regeneration vary from species to species of plants, but generally a suspension of transformed protoplasts containing copies of the heterologous gene is first provided. Callus tissue is formed and shoots may be induced from callus and subsequently rooted.

Alternatively, embryo formation can be induced from the protoplast suspension. These embryos germinate and form mature plants. The culture media will generally contain various amino acids and hormones, such as auxin and cytokinins. Shoots and roots normally develop simultaneously. Efficient regeneration will depend on the medium, on the genotype, and on the history of the culture. The reproducibility of regeneration depends on the control of these variables.

Transgenic lines are established from transgenic plants by tissue culture propagation. The presence of nucleic acid sequences encoding a RNAi gene of the present invention (including mutants or variants thereof) may be transferred to related varieties by traditional plant breeding techniques.

### II. Providing and Breeding Transgenic Maize Plants.

Transgenic plants of the present inventions are provided from plant material such as meristem primordia tissue that was transformed with plasmids, each containing a particular heterologous gene expression cassette (such as an RANi construct) using the Biolistic bombardment method as described in Example 5 and in U.S. Pat. No. 5,767,368 to Zhong et al. Further examples of the Biolistic bombardment method are disclosed in U.S. application Ser. No. 08/036,056 and U.S. Pat. No. 5,736,369 to Bowen et al. Each heterologous gene expression cassette is separately introduced into a plant tissue and the transformed tissue propagated to produce a transgenic plant that contains the particular heterologous RNAi gene expression cassette. Thus, the result is a transgenic plant containing the heterologous RNAi gene expression cassette expressing a silencing construct for a lignin enzyme, such as CCL, CAD, etc., and a transgenic plant containing a heterologous gene expression cassette expressing a selection enzyme, such as bar.

Alternatively, transformation of corn plants can be achieved using electroporation or bacterial mediated transformation using a bacterium such as Agrobacterium tumefaciens to mediate the transformation of corn root tissues (see Valvekens et al. Proc. Nat'l. Acad. Sci. USA. 85: 5536 5540 (1988), herein incorporated by reference) or meristem primordial as described herein.

In a preferred embodiment of the present invention, the transgenic plant comprises one or more silencing genes for a lignin enzyme protein and one or more selection markers. Construction of the preferred transgenic plant comprises making first generation transgenic plants as above, each comprising a silencing gene construct, and transgenic plants as above, each comprising a silencing gene. After each first generation transgenic plant has been constructed, progeny from each of the first generation transgenic plants are cross-bred by sexual fertilization to produce second generation transgenic plants comprising various combinations of both a silencing gene and commercially desired traits. In a further embodiment, transgenic plants with one silencing gene are cross-bred to transgenic plants comprising other silencing genes. For example, various combinations of progeny from the first generation transgenic plants are crossbred to other corn plants in order to produce second generation transgenic showing a reduction in lignin content. Progeny of the second generation transgenic plants are cross-bred by sexual fertilization among themselves or with first generation transgenic plants to produce third generation transgenic plants that contain one or more silenced genes, or combinations thereof. For example, cross-breeding a second generation transgenic plant with a reduction in CCD expression with a second generation transgenic plant with a reduction in CAD expression produces a third generation transgenic plant with reduced aligning that is economically desirable for producing ethanol.

In other embodiments, transgenic plants with various combinations of reduced lignin enzymes can be made by cross-breeding progeny from a particular transgenic plants. For example, Zhang et al, Theor. Appl. Genet. 92: 752 761, (1996), Zhong et al, Plant Physiol. 110: 1097 1107, (1996), and Zhong et al, Planta, 187: 483 489, (1992), herein incorporated by reference, provide methods for making transgenic plants by sexual fertilization.

Alternatively, plant material is transformed as above with a plasmid containing a heterologous RNAi expression cassette encoding silencing constructs. The transgenic plant is recovered from the progeny of the transformed plant material. Next, plant material from the transgenic plant is transformed with a second plasmid containing a heterologous RNAi gene expression cassette encoding a different silencing gene and a second selectable marker. The transgenic plant is recovered from the progeny of the transformed plant material. Such that any desired combination of silenced genes in transgenic corn plants are contemplated.

In a preferred embodiment, the above heterologous RNAi gene expression cassettes further include therein nucleotide sequences that encode one or more selectable markers which enable selection and identification of transgenic plants that express the modified lignin of the present invention. Preferably, the selectable markers confers additional benefits to the transgenic plant such as herbicide resistance, insect resistance, and/or resistance to environmental stress.

Alternatively, the above transformations are performed by co-transforming the plant material with a first plasmid containing a gene expression cassette encoding a selectable marker and a second plasmid containing a heterologous RNAi gene expression cassette encoding a silencing construct. The advantage of using a separate plasmid is that after transformation, the selectable marker can be removed from the transgenic plant by segregation, which enables the selection method for recovering the transgenic plant to be used for recovering transgenic plants in subsequent transformations with the first transgenic plant.

Examples of preferred markers that provide resistance to herbicides include, but are not limited to, the bar gene from Streptomyces hygroscopicus encoding phosphinothricin acetylase (PAT), which confers resistance to the herbicide glufonsinate; mutant genes which encode resistance to imidazalinone or sulfonylurea such as genes encoding mutant form of the ALS and AHAS enzyme as described by Lee at al. EMBO J. 7: 1241 (1988) and Miki et al., Theor. Appl. Genet. 80: 449 (1990), respectively, and in U.S. Pat. No. 5,773,702, all of which are herein incorporated by reference genes which confer resistance to glycophosphate such as mutant forms of EP SP synthase and aroA; resistance to L-phosphinothricin such as the glutamine synthetase genes; resistance to glufosinate such as the phosphinothricin acetyl transferase (PAT and bar) gene; and resistance to phenoxy proprionic acids and cycloshexones such as the ACCAse inhibitor-encoding genes (Marshall et al. Theor. Appl. Genet. 83: 435 (1992), herein incorporated by reference). The above list of genes which can import resistance to an herbicide is not inclusive and other genes not enumerated herein but which have the same effect as those above are within the scope of the present invention.

Examples of preferred genes which confer resistance to pests or disease include, but are not limited to, genes encoding a Bacillus thuringiensis protein such as the delta-endotoxin, which is disclosed in U.S. Pat. No. 6,100,456, herein incorporated by reference; genes encoding lectins, (Van Damme et al., Plant Mol. Biol. 24: 825 (1994), herein incorporated by reference); genes encoding vitamin-binding proteins such as avidin and avidin homologs which can be used as larvicides against insect pests; genes encoding protease or amylase inhibitors, such as the rice cysteine proteinase inhibitor (Abe et al., J. Biol. Chem. 262: 16793 (1987), herein incorporated by reference) and the tobacco proteinase inhibitor I (Hubb et al., Plant Mol. Biol. 21: 985 (1993)); genes encoding insect-specific hormones or pheromones such as ecdysteroid and juvenile hormone, and variants thereof, mimetics based thereon, or an antagonists or agonists thereof; genes encoding insect-specific peptides or neuropeptides which, upon expression, disrupts the physiology of the pest; genes encoding insect-specific venom such as that produced by a wasp, snake, etc.; genes encoding enzymes responsible for the accumulation of monoterpenes, sesquiterpenes, asteroid, hydroxaminc acid, phenylpropanoid derivative or other non-protein molecule with insecticidal activity; genes encoding enzymes involved in the modification of a biologically active molecule (see U.S. Pat. No. 5,539,095 to Sticklen et al., which discloses a chitinase that functions as an anti-fungal); genes encoding peptides which stimulate signal transduction; genes encoding hydrophobic moment peptides such as derivatives of Tachyplesin which inhibit fungal pathogens; genes encoding a membrane permease, a channel former or channel blocker (for example cecropin-beta lytic peptide analog renders transgenic tobacco resistant to Pseudomonas solanacerum) (Jaynes et al. Plant Sci. 89: 43 (1993)); genes encoding a viral invasive protein or complex toxin derived therefrom (viral accumulation of viral coat proteins in transformed cells of some transgenic plants impart resistance to infection by the virus the coat protein was derived as shown by Beachy et al. Ann. Rev. Phytopathol. 28:451 (1990); genes encoding an insect-specific antibody or antitoxin or a virus-specific antibody (Tavladoraki et al. Nature 366: 469 (1993), herein incorporated by reference); and genes encoding a developmental-arrestive protein produced by a plant, pathogen or parasite which prevents disease. The above list of genes which can import resistance to disease or pests is not inclusive and other genes not enumerated herein but which have the same effect as those above are within the scope of the present invention.

Examples of genes which confer resistance to environmental stress include, but are not limited to, mtld and HVA1, which are genes that confer resistance to environmental stress factors; rd29A and rd19B, which are genes of Arabidopsis thaliana that encode hydrophilic proteins which are induced in response to dehydration, low temperature, salt stress, or exposure to abscisic acid and enable the plant to tolerate the stress (Yamaguchi-Shinozaki et al., Plant Cell 6: 251-264 (1994)). Other genes contemplated can be found in U.S. Pat. Nos. 5,296,462 and 5,356, herein incorporated by reference. The above list of genes, which can import resistance to environmental stress, is not inclusive and other genes not enumerated herein but which have the same effect as those above are within the scope of the present invention.

Thus, it is within the scope of the present invention to provide transgenic plants which express one or more silencing genes, and one or more of any combination of genes which confer resistance to an herbicide, pest, or environmental stress.

In particular embodiments of the present invention, the heterologous RNAi gene expression cassettes can further be flanked with DNA containing the matrix attachment region (MAR) sequence. While use of MAR in the present invention is optional, it can used to increase the expression level of transgenes, to get more reproducible results, and to lower the average copy number of the transgene (Allen et al., The Plant Cell 5:603-613 (1993); Allen et al., The Plant Cell 8: 899-913 (1996); Mlynarova et al., The Plant Cell 8: 1589-1599 (1996), herein incorporated by reference).

III. Production of Glucose from Lignocellulosic Biomass.

The major products of the biofuels industry are ethanol and biodiesel. Ethanol makes up over 90 percent of current US biofuels production of about 4 billion gallons per year.

Currently, ethanol production requires corn or other high-starch grains, water, chemicals, enzymes and yeast, and denaturants such as unleaded gas.

In the dry milling process (used for about 80 percent of production), corn or other high-starch grains are first ground into meal and then mixed with water and enzymes to form a mash. The mash is processed at a high temperature in cookers to liquefy the mixture and reduce bacteria levels prior to fermentation. Next, the mash is cooled and secondary enzymes added to convert the starches into glucose sugars. Yeast and ammonia are added to the mash and the mixture is passed through several fermenters, completing the process of converting the sugar to ethanol and carbon dioxide. After fermentation, the fermented mash, which is about 10 percent alcohol, is transferred to distillation, where the ethanol is separated from the residual solids. The ethanol is concentrated to 190 proof using conventional distillation methods, and then dehydrated to approximately 200 proof (100 percent alcohol). The resulting ethanol is then blended with about 5 percent denaturant, usually gas, to prevent human consumption, and is then ready for shipment to a blending site. The residual solids are processed and sold as high-protein animal feed.

In the wet milling process (used for about 20 percent of ethanol production), the grain is first steeped in a dilute sulphuric acid to facilitate separation of the grain into its component parts. The mixture is then ground, the germ separated, and enzymes added to convert the starches to glucose. After fermentation and distillation of the ethanol, the remaining mash is recombined with fiber and sold as corn gluten, an animal feed.

For determining the success of lignin reduction, the inventor contemplates degrading the lignocellulose in the leaves and stalks of the transgenic plants of the present invention, by grinding up the stover to produce a corn biomass. The lignocellulose of the transgenic plant would be processed into fermentable sugars, primarily glucose, and residual solids. The fermentable sugars are contemplated for use to produce ethanol or other products.

The transgenic plants can be processed to ethanol in an improvement on the separate saccharification and fermentation (SHF) method (Wilke et al., Biotechnol. Bioengin. 6: 155-175 (1976)) or the simultaneous saccharification and fermentation (SSF) method disclosed in U.S. Pat. No. 3,990,944. and U.S. Pat. No. 3,990,945, herein incorporated by reference. The SHF and SSF methods require pre-treatment of the plant material feedstock with dilute acid to make the cellulose more accessible followed by enzymatic hydrolysis using exogenous cellulases to produce glucose from the cellulose, which is then fermented by yeast to ethanol. In some variations of the SHF or SSF methods, the plant material is pre-treated with heat or with both heat and dilute acid to make the cellulose more accessible.

Exemplary pretreatment and Enzymatic Hydrolysis assays contemplated for use in evaluating transgenic maize lines of the present inventions. Milled transgenic lignin down regulated maize versus wild-type maize stover (about 1 cm in length) was pretreated using the AFEX technology (see inventor patent: U.S. Pat. No. 7,049,485. Transgenic plants containing ligninase and cellulase which degrade lignin and cellulose to fermentable sugars. Issues, Jun. 1, 2006). In more detail, the transgenic versus wild-type maize biomass was transferred to a high-pressure reactor (PARR Instrument Col, IL) with 60% moisture (kg water/kg dry biomass) and liquid ammonia ratio 1.0 (kg of ammonia/kg of dry biomass) was added. The temperature was slowly raised and the pressure in the vessel increased. The temperature was maintained at 90° C. for five minutes before explosively releasing the pressure. The instantaneous drop of pressure in the vessel caused the ammonia to vaporize, causing an explosive decompression and considerable fiber disruption. The pretreated material was kept under a hood to remove residual ammonia and stored in a freezer until further use. Then commercial cellulases were added for enzymatic hydrolysis. Finally, the amount of fermentable sugars produced from transgenic versus wild-type maize stover was compared. An increase in level of fermentable sugars in lignin down regulated transgenic plants meant less needs for pretreatment processes.

The enzyme hydrolysis was performed in a sealed scintillation vial. A reaction medium, composed of 7.5 ml of 0.1 M, pH 4.8 sodium citrate buffer, was added to each vial. In addition, 60 μl (600 μg) tetracycline and 45 μl (450 μg) cycloheximide were added to prevent the growth of microorganisms during the hydrolysis reaction. The pretreated corn stover substrate was hydrolyzed using commercial cellulase enzymes at a glucan loading of 1% (w:v) biomass. Distilled water was then added to bring the total volume in each vial to 15 ml. All reactions were performed in duplicate to test reproducibility. The hydrolysis reaction was carried out at 50° C. with a shaker speed of 90 rpm. About 1 ml of sample was collected at 72 hr of hydrolysis, filtered using a 0.2 mm syringe filter and kept frozen. The amount of glucose produced in the enzyme blank and substrate blank were subtracted from the respective hydrolyzed glucose levels. Hydrolyzate was quantified using Waters HPLC by running the sample in Aminex HPX-87P (Biorad) column, against sugar standards.

In some embodiments the present invention contemplates that transgenic corn lines with reduced lignin content will provide advantages over other plants, including providing corn plants with viable cell walls, plant structural integrity, and a higher level of glucose conversion with less cost.

### EXPERIMENTAL

The following examples serve to illustrate certain embodiments and aspects of the present invention and are not to be construed as liming the scope thereof.

In the experimental disclosures which follow, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); pg (picograms); L and l (liters); ml (milliliters); μl (microliters); cm (centimeters); mm (millimeters); μm (micrometers); nm (nanometers); U (units); min (minute); s and sec (second); k (kilometer); deg (degree); ° C. (degrees Centigrade/Celsius), colony-forming units (cfu), optical density (OD), and polymerase chain reaction (PCR).

### Example I

This example describes exemplary materials and methods for assays used during the development of the present inventions.

### Lignin Analysis.

Briefly, sections from the base of mature stems are lyophilised, ground and analysed by different methods, Halpin et al. (1998) The Plant Journal, 14:545; herein incorporated by reference. Klason determinations were performed on the dried insoluble cell wall residue (CWR) of samples soxhlet extracted with toluene/ethanol, ethanol and water according to the method of Effland, 1977, TAPPI 6: (10); herein incorporated by reference). CWR (300 mg) was treated with 3 ml 72% w/v sulphuric acid (2 h; 20° C.) then diluted to 5% acid and boiled under reflux (3 h). The sample was filtered in a tared n° l porosity glass cinter, washed, dried (100° C.; 20 h) and weighed. Thioacidolysis was performed on 10 mg CWR using 0.2N BF₃ etherate in 8 ml of dioxane-ethanethiol (v/v, 9/1). After 4 h at 100° C., monomeric products released from lignin were analysed, as their trimethylsililates, by gas chromatography according to Lapierre et al., (1986), Holzforschung 40:113-118; which is herein incorporated by reference.

### Polymerase Chain Reaction Confirmation of Transgenic Plant Cells Based Upon the Presence of a Bar Gene and the RNAi Construct Using RNAi Primer Sequences.

The inventor confirmed the presence of the RNAi construct in corn plant cells using conventional PCR methods. DNA amplifications were performed in a thermo cycler (PerkinElmer/Applied Biosystem, Foster City, Calif.) using initial denaturation at 94° C. for 4 min, followed by 35 cycles of 1 min at 94° C., 1 min at 55° C., 2 min at 72° C., and a final 10 min extension at 72° Celcius. The reaction mixture was loaded directly onto a 0.8% (w/v) agarose gel, stained with ethidium bromide, and visualized with UV light. In some cases the PCR products of the specific transgene were used in Northern blots for determining relative levels of gene expression.

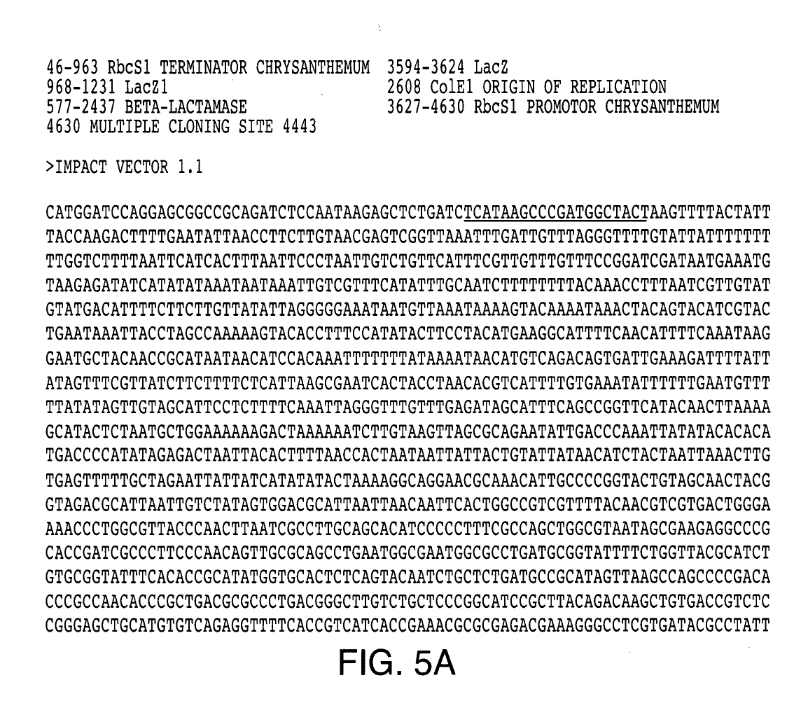

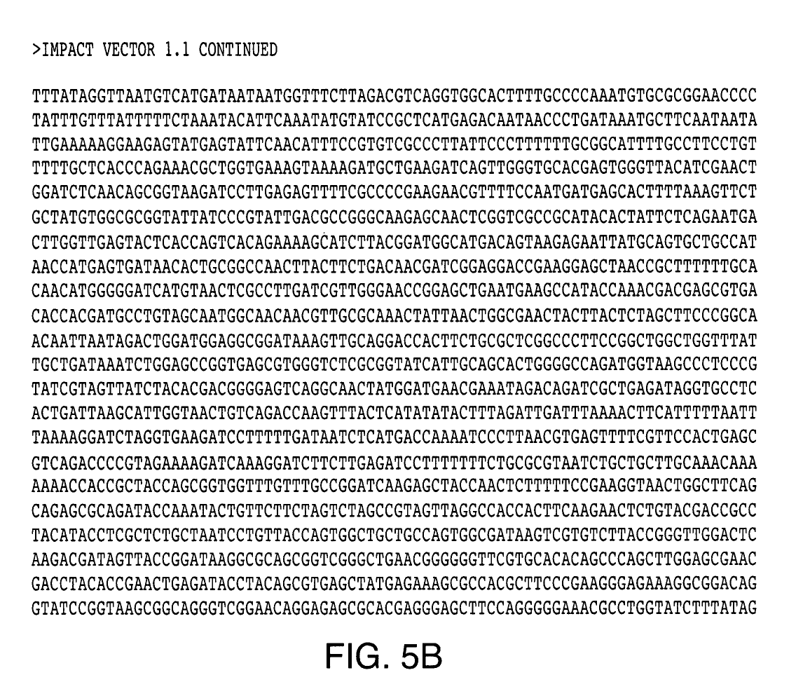

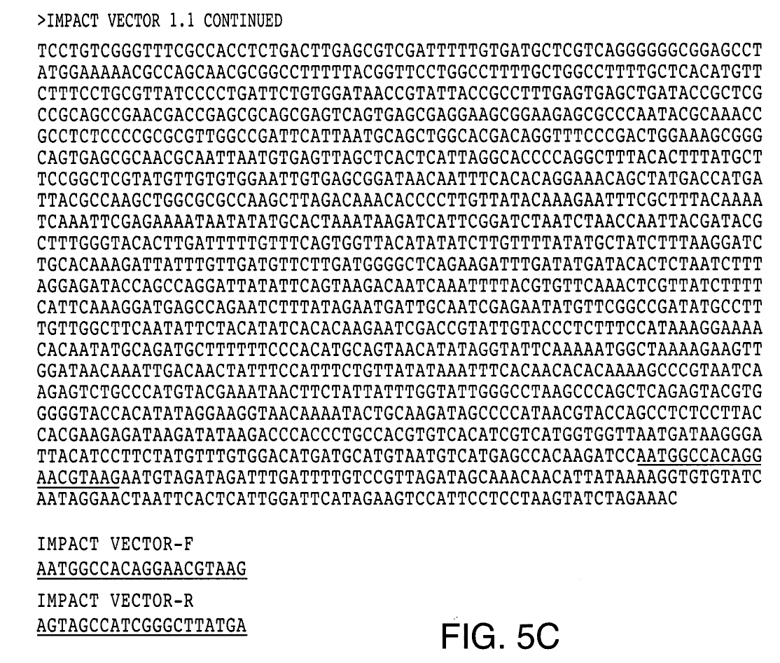

Specifically, bar primers used were 5′-ATG AGC CCA GAA CGA CG-3′ (forward primer); bar R, 5′-TCA GAT CTC GGT GAC GG-3′ (reverse primer). The transgene product size was about 0.59 kb for the bar gene (Oraby, et al., 2005, Crop Sci 45:2218-2227, herein incorporated by reference). RNAi was detected for ImpactVector 1.1 with the primers shown in FIG. 5.

### RNA Isolation and Northern Blot Hybridization Analysis.

Assays for showing the presence of a bar gene, the RNAi vector and loss of lignin enzyme expressing genes were also performed by standard Northern blots. Total RNA was isolated from young leaves of corn plants (transgenic and nontransgenic) using the TRI Reagent (Sigma-Aldrich, St. Louis, Mo.) according to the manufacturer's instructions. For the Northern blot, 20 μg of RNA were separated in a 1.2% (w/v) agarose-formaldehyde denaturing gels according to Sambrook et al. (1989) and blotted onto Hybond-N+ nylon membranes (Amersham-Pharmacia Biotech). Gene expression was analyzed by a standard Northern-blotting method (Sambrook et al., 1989) using the coding sequence as a probe labeled with -[32P]-dCTP with the Random Primer Labeling Kit (Invitrogen, Carlsbad, Calif.) according to the manufacturer's instructions (Oraby, et al., 2005, Crop Sci 45:2218-2227, herein incorporated by reference).

Exemplary compositions and methods for measuring levels of digestibility of corn stover, and amount of pretreatment (such as Ammonia Fiber Expansion (AFEX)) as compared to the control wild type are described in (Dale et al., Biosource technol. 56: 11 116 (1996), herein incorporated by reference). The inventor contemplates that samples of the transgenic maize plants, such as stover, of the present inventions would be treated by the ammonia fiber explosion process in order to measure cellulose and hemicellulose accessibility. As used herein, “corn stover” refers to a composition of leaves, stalks and cobs of maize plants, such as those left in the field after harvest of corn grain.

### Example II

This example describes exemplary compositions and methods for obtaining cDNAs of the corn lignin biosynthesis enzymes and RNAi constructs of the present inventions.

An ImpactVector™ for cytoplasmic expression, comprising a light-regulated Asteraceous chrysanthemum Ribulose bisphosphate carboxylase (RBC) (RbcS1 (rubisco)) promoter sequence, Outchkourov et al. 2003, was used for integrating RNAi constructs and transforming corn tissue. This vector comprises a universal multiple cloning site, Plant Research International of Wageningen University and Research Center (ww.pri.wur.nl/UK/products/ImpactVector).

The ImpactVector™ family enables targeting of a protein into one of 5 different subcellular compartments, of which the inventor choose cytoplasmic expression of the 1.1 vector (FIG. 5).

The plasmid BY520 contained the linked selectable marker/herbicide resistance bar (phosphinothricin acetyl transferase) gene (driven by cauliflowermosaic virus 35S promoter and the nopaline synthase nos terminator).

The plasmid pDM302 was described in U.S. Pat. No. 7,049,485 and Cao et al., Plant Cell Reports 11: 586 591 (1992), all of which are herein incorporated by reference),

### Example III

This example describes exemplary compositions and methods for providing co-transformed corn plants with the each of the RNAi constructs described in EXAMPLE II, FIG. 4, and a construct comprising a bar herbicide resistance gene regulated under an separate plant-specific promoter. Specifically, this example shows the transformation of maize multi-meristem primordia via Biolistic bombardment with the plasmid constructs of FIG. 4, regeneration of the transgenic plants, confirmation of the integration of the plasmid constructs into the plant genome, and confirmation of the expression of the cellulase or ligninase fusion proteins in the transgenic plant. For transformations with the constructs which do not contain a selectable marker, a selectable marker comprising the bar gene in the plasmid pDM302 (Cao et al., Plant Cell Reports 11: 586 591 (1992), herein incorporated by reference) is cotransfected into the cells with the plasmid containing the ligninase or cellulase heterologous gene expression cassette.

Maize seeds were germinated in Murashige and Skoog (MS) medium (Murashige and Skoog, Physiol. Plant 15: 473 497 (1962), herein incorporated by reference) supplemented with the appropriate growth regulators (Zhong et al., Planta 187: 490 497 (1992), herein incorporated by reference). Shoot meristems were dissected and cultured for 2 3 weeks until an initial multiplication of meristem have been produced for bombardment.

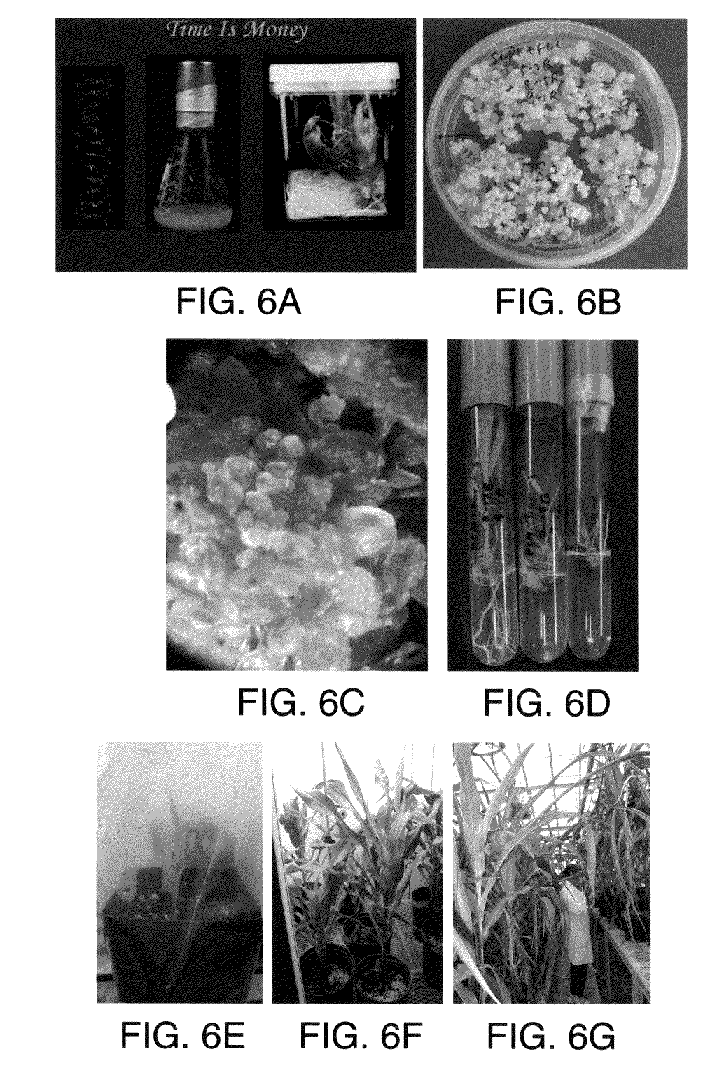

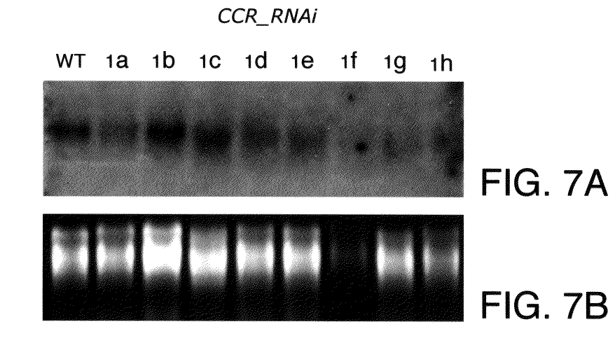

Corn transformation, acclimation and transfer to greenhouses: Quickly proliferating, immature-embryo-derived Type II embryogenic callus (FIGS. 6B and C) was produced and used in transformation experiments. The multi-meristem primordia explants are bombarded with tungsten particles coated with RNAi constructs cloned into plasmids, such as ImpactVector1.1, along with the plasmid containing the heterogenous gene expression cassette containing the bar gene. The bombarded explants are gently transferred onto meristem multiplication medium for further multiplication, about 6 to 8 more weeks. This step is required to reduce the degree of chimerism in transformed shoots prior to their chemical selection. Two to four hours prior to bombardment, callus was transferred in penny size circles in center of a Petri dish containing an osmotic or conditioning medium. Conditioned callus was bombarded with ethanol washed tungsten particles combined with a total of 10 μg of 1:1 mixture of each RNAi construct and the pDM302 according to the manufacturer's protocol (BioRad PDS 1000/He® Biolistic gun) at a pressure of 1100 pounds per square inch (PSI). Shoots are transferred to the above medium containing 5 to 10 mg per liter glufosinate ammonium (PPT) selectable chemical for another 6 to 8 weeks. Chemically selected shoots are rooted in rooting medium containing the same concentration of PPT. Plantlets are transferred to pots, acclimated, and then transferred to a greenhouse.

When the plantlets or shoots are small, the quantity of transgenic plant material is insufficient for providing enough DNA for Southern blot hybridization; therefore, polymerase chain reaction (PCR) is used to confirm the presence of the plasmid constructs in the plantlets. The amplified DNA produced by PCR is resolved by agarose or acrylamide gel electrophoresis, transferred to membranes according standard Northern transfer methods, and probed with the appropriate DNA construct or portion thereof according to standard Northern hybridization methods. Those shoots or plantlets which show they contain the construct in its proper form are considered to have been transformed. The transformed shoots or plantlets are grown in the greenhouse to produce sufficient plant material to confirm that the plasmid construct was properly integrated into the plant genome. To confirm proper integration of the plasmid constructs into the plant genome, genomic DNA is isolated from the greenhouse grown transgenic plants and untransformed controls and analyzed by standard Southern blotting methods as in Zhong et al., Plant Physiology 110: 1097 1107 (1996); Zhang et al., Theor. Appl. Genet. 92: 752 761 (1996); Zhang et al., Plant Science 116: 73 84 (1996); and, Jenes et al., In Transgenic Plants. Vol. 1. Kung, S-D and Wu, R (eds.). Academic Press, San Diego, Calif. pp. 125 146 (1992), herein incorporated by reference.

The pDM302 and pBY520 plasmids were used to provide the selectable marker herbicide resistance. Multiple shoot meristems were also produced following the methods provided in U.S. Pat. No. 5,767,368. Method for producing a cereal plant with foreign DNA. Issued Jun. 16, 1998; and publication: Zhong, et al., (1992) Morphogenesis of corn (Zea mays L.) in vitro I. Formation of multiple shoot clumps and somatic embryos from shoot tips. Planta 187: 490-497, all of which are herein incorporated by reference). The bombarded callus or multimeristem was kept on the same conditioning medium for 24 hours, transferred to callus proliferation medium for five days or to multimeristem shoot elongation medium.

At least 40 transgenic plants tested positive for RNAi vectors comprising CCR, CAD and 4CL, showing various levels of reduced transcription of the targeted gene.

### Example IV

This example describes exemplary compositions and methods for the selection of transgenic lines which are resistant to Phosphinotricin (PPT) (for bar) in vitro.

Cell lines or multiplemeristem lines were placed on selection medium containing 2 mg/L Bialaphos where they were maintained for six to eight weeks with a two-week subcultures into fresh medium. Cultures were maintained in the dark up to this point. The detected Bialaphos-resistant surviving callus clones or multiple meristems were placed in regeneration medium and exposed to light (60 μmol quanta m-²·s-¹ from cool-white 40 W Econ-o-watt fluorescent lamps; Philips Westinghouse, USA) for four to six weeks. Plantlets were transferred further to rooting medium containing 2 mg/L Bialaphos selectable herbicide, and maintained for two to four weeks under the above light conditions. Rooted plantlets of eight to ten cm in height were transferred to pots containing soil, and pots were covered with plastic bags and kept under light to mimic the tissue culture conditions. Small holes were made daily in each bag, for 10-14 days acclimating the plants to greenhouse conditions.

Rooted acclimated plants were transplanted into two-gallon pots and transferred to a long day (16 hours/day light) greenhouses. When long day was not available in greenhouses, they were transferred to walk-in growth chambers with the same conditions.

### Example V

This example describes exemplary compositions and methods for comparing the transcription levels of RNAi transgenic corn leaves of the present inventions with the transcription of wild type corn leaves.

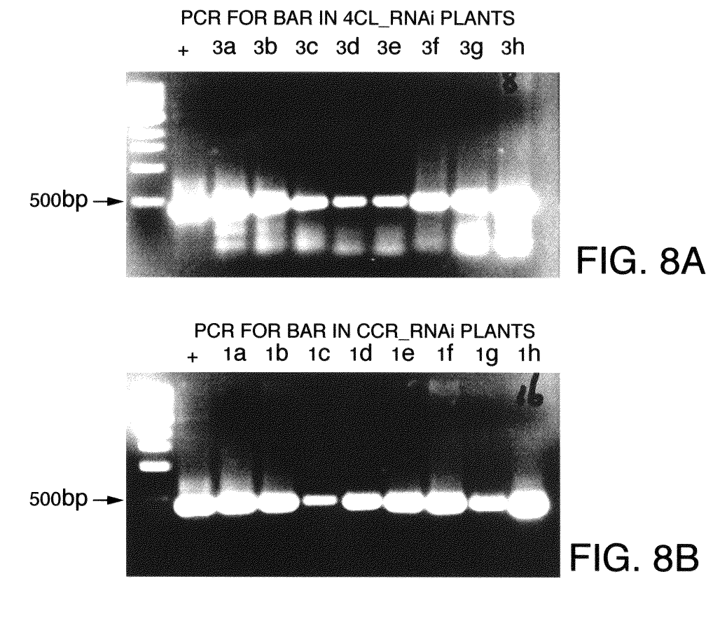

RNA was collected as described herein. A loading value of 15 ug of RNA per lane was used for this Northern blot analysis. Although numerous transgenic lines were developed, certain lines were chosen for a reduction in target gene expression. For example, Transgenic line numbers e, f, g, h in FIG. 8 demonstrate a lower level of transcription.

### Example VI

This example describes exemplary compositions and methods for breeding commercially acceptable lines of promising transgenic corn plants by using self breeding and cross breeding methods.

Lignin down regulated and wild-type maize plants grown in greenhouses were routinely checked for production of tassels, mature pollens (male cells) and kernels (females). As soon as pollens were matured, the kernel husks were opened; the pollens were brushed on top of the silk of kernels. Then, kernel husks were closed and the mated kernel was placed inside a maize breeding paper bag, and information was written on the bag. When kernels produced mature seeds, the kernels were harvested and stored in a cold room for further experimentations. In the breeding, the male and female were selected from the same plants when male and female were available in the same plant. This practice was called self-breeding. Alternatively, transgenic plants with different lignin enzymes down regulation were cross-bred for production of hydride maize. For example the male of CCR down regulated plant was mated with the female of a 4CL down regulated plant. These lines are under evaluation for commercial use, including as sources of lignin biomass for glucose production and further for use in alcohol production.

All publications and patents mentioned in the above specification are herein incorporated by reference in their entirety. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, plant biology, plant disease, agriculture, biofuels, biochemistry, chemistry, and plant pathogens or related fields are intended to be within the scope of the following claims. All references cited herein are incorporated in their entirety.

We claim:

1. An expression vector, wherein said vector comprises a first nucleotide, wherein said first nucleotide interferes with a second nucleotide encoding a polypeptide that alters lignin biosynthesis, and wherein said first nucleotide is in operable combination with a riburose-1,5-bisphosphate carboxylase small-subunit (RbcS1) promoter.

2. The expression vector of claim 1, wherein said vector expresses said first nucleotide in cytoplasm.

3. The expression vector of claim 1, wherein said promoter is a Chrysanthemum promoter.

4. The expression vector of claim 1, wherein said altered lignin biosynthesis reduced lignin biosynthesis.

5. The expression vector of claim 1, wherein said altered lignin biosynthesis modifies a lignin structure.

6. The expression vector of claim 1, wherein said polypeptide is selected from the group consisting of a phenyl ammonia lyase; cinnamate 4-hydroxylase; para-coumarate 3-hydroxylase; S-adenosylmethione:caffeate/5-hydroxyferulate-O-methyltransferase caffeic acid O-methyltransferase; caffeoyl-CoA O-methyltransferase CCoAOMT; 4-coumarate:CoA ligase; cinnamoyl-CoA reductase; cinnamyl alcohol dehydrogenase; sinapyl alcohol dehydrogenase; para-hydroxycinnamoyl-CoA:quinate shikimate para-hydroxycinnamoyltransferase; ferulate 5-hydroxylase; homologs and orthologs thereof.

7. The expression vector of claim 1, wherein said second nucleotide is selected from the group consisting of SEQ ID NOs:1-5.

8. The expression vector of claim 1, wherein said polypeptide is selected from the group consisting of SEQ ID NOs:6-10.

9. The expression vector of claim 1, wherein said first nucleotide comprises an RNAi molecule selected from the group consisting of a siRNA, hairpin siRNA, miRNA and snRNA.

10. The expression vector of claim 1, further comprising an RNAi construct, wherein said construct comprises said first nucleotide sequence in an antisense direction in operable combination with said first nucleotide sequence in a sense direction.

11. A transgenic maize cell, wherein said maize cell comprises an RNAi gene silencing construct in operable combination with a riburose-1,5-bisphosphate carboxylase small-subunit (RbcS1) promoter.

12. The transgenic maize cell of claim 11, wherein said RNAi construct comprises an oligonucleotide in an antisense direction in operable combination with said oligonucleotide in a sense direction.

13. The transgenic maize cell of claim 12, wherein said oligonucleotide comprises a portion of a first nucleotide sequence encoding a polypeptide selected from the group consisting of a phenyl ammonia lyase; cinnamate 4-hydroxylase; para-coumarate 3-hydroxylase; S-adenosylmethione:caffeate/5-hydroxyferulate-O-methyltransferase caffeic acid O-methyltransferase; caffeoyl-CoA O-methyltransferase CCoAOMT; 4-coumarate:CoA ligase; cinnamoyl-CoA reductase; cinnamyl alcohol dehydrogenase; sinapyl alcohol dehydrogenase; para-hydroxycinnamoyl-CoA:quinate shikimate para-hydroxycinnamoyltransferase; ferulate 5-hydroxylase; homologs and orthologs thereof.

14. The transgenic maize cell of claim 12, wherein said oligonucleotide comprises at least a portion of a second nucleotide sequence selected from the group consisting of SEQ ID NOs:1-5.

15. The transgenic maize cell of claim 11, wherein said maize cell comprises a plant part.

16. A composition comprising a transgenic maize cell, wherein said maize cell comprises an RNAi gene silencing construct in operable combination with a riburose-1,5-bisphosphate carboxylase small-subunit (RbcS1) promoter.

17. A method of gene silencing in a maize plant part, comprising,
a) providing,
i) a maize plant part,
ii) a nucleic acid sequence encoding an enzyme, wherein said enzyme alters a lignin structure,
iii) a gene silencing construct, wherein said RNAi gene silencing construct is in operable combination with a riburose-1,5-bisphosphate carboxylase small-subunit (RbcS1) promoter, and
b) transfecting said gene silencing construct into said plant part for silencing said enzyme.

18. The method of claim 17, wherein said silencing alters lignin production while retaining the desired characteristics of a plant cell wall.

19. A method for producing glucose from a lignocellulosic biomass, comprising,
a) providing,
i) a lignocellulosic biomass, comprising an RNAi gene silencing construct in operable combination with a riburose-1,5-bisphosphate carboxylase small-subunit (RbcS1) promoter, and
ii) a composition capable of converting cellulose to glucose,
b) converting said lignocellulosic biomass into glucose using said composition.

20. The method for producing glucose of claim 19, wherein said lignocellulosic biomass comprises maize corn stover.

21. The method for producing glucose of claim 19, wherein composition is selected from the group consisting of an ammonia fiber and a hydrolytic enzyme.


SEQUENCE LISTING (25 sequences)

SEQ ID NO: 1 - DNA, 1481 bp, Zea mays
ctcgatcgtcgtcatcacgctcgaccgcacaactgcaccaaggggggaggagacctaaaaactactacatcttttagctacacatctagctaaagatcgagaggggtaaataaggacgagcgggcgcgagctagaagagcagctgcaggtactaccatcatcgtcgtcgtcgtcgccaggatgaccgtcgtcgacgccgtcgtctcctccaccgatgccggcgcccctgctgccgccgccaccgcggtaccggcggggaacgggcagaccgtgtgcgtgaccggcgcggccgggtacatcgcctcgtggttggtgaagctgctgctcgagaagggatacactgtgaagggcaccgtcaggaacccagatgacccgaagaacgcgcacctcaaggcgctggacggcgccgccgagcggctgatcctctgcaaggccgatctgctggactacgacgccatctgccgcgccgtgcagggctgccagggcgtcttccacaccgcctcccccgtcaccgacgacccggagcaaatggtggagccggcggtgcgcggcaccgagtacgtgatcaacgcggcggcggaggccggcacggtgcggcgggtggtgttcacgtcgtccatcggcgccgtgaccatggaccccaagcgcgggcccgacgtcgtggtcgacgagtcgtgctggagcgacctcgagttctgcgagaaaaccaggaactggtactgctacggcaaggcggtggcggagcacgcggcgtgggagacggcccggcggcggggcgtggacctggtggtggtgaaccccgtgctggtggtgggccccctgctgcaggcgacggtgaacgccagcatcgcgcacatcctcaagtacctggacggctcggcccgcaccttcgccaacgccgtgcaggcgtacgtggacgtgcgcgacgtggccgacgcgcacctccgcgtcttcgagagcccccgcgcgtccggccgccacctctgcgccgagcgcgtcctccaccgcgaggacgtcgtccgcatcctcgccaagctcttccccgagtaccccgtcccagccaggtgctccgacgaggtgaatccgcggaagcagccgtacaagttctccaaccagaagctccgggacctggggctgcagttccggccggtcagccagtcgctttacgacacggtgaagaacctccaggagaagggacacctgccggtgctcggagagcggacgacgacggaggccgccgacaaggatgcccccacggccgagatgcagcagggagggatcgccatccgtgcctgagagggcgatgccacacatgaacacaaagcaatgttcatactgctgccctgcacctgctgtgtaaacaggcctgtgtttgttctggctgatagtgatgtaccctaagacttgtaacgtcatgttcgttcttgtgaactatagcgagtgaataaaattggttaatgttggatgttcaaaaaaaaaaa

SEQ ID NO: 2 - DNA, 1668 bp, Zea mays
  misc_feature (603)..(603): n is a, c, g, or t
atgggttccgtagacgcggcgatcgcggtgccggtgccggcggcggaggagaaggcggtggaggagaaggcgatggtgttccggtccaagcttcccgacatcgagatcgacagcagcatggcgctgcacacctactgcttcgggaagatgggcgaggtggcggagcgggcgtgcctgatcgacgggctgacgggcgcgtcgtacacgtacgcggaggtggagtccctgtcccggcgcgccgcgtcggggctgcgcgccatgggggtgggcaagggcgacgtggtgatgagcctgctccgcaactgccccgagttcgccttcaccttcctgggcgccgcccgcctgggcgccgccaccaccacggccaacccgttctacaccccgcacgaggtgcaccgccaggcggaggcggccggcgcccggctcatcgtgaccgaggcctgcgccgtggagaaggtgcgggagttcgcggcggagcggggcatccccgtggtcaccgtcgacgggcgcttcgacggctgcgtggagttcgccgagctgatcgcggccgaggagctggaggctgacgccgacatccaccccgacgacgtcgtcgcgctgccntactcctccggcaccaccgggctgcccaagggcgtcatgctcacccaccgcagcctcatcaccagcgtcgcgcagcaggttgatggcgagaacccgaacctgtacttccgcaaggacgacgtggtgctgtgcctgctgccgctgttccacatctactcgctgaactcggtgctgctggccggcctgcgcgcgggctccaccatcgtgatcatgcgcaagttcgacctgggcgcgctggttgacctggtgcgcaggtacgtgatcaccatcgcgcccttcgtgccgcccatcgtggtggagatcgccaagagcccccgcgtgaccgccggcgacctcgcgtccatccgcatggtcatgtccggcgccgcgcccatgggcaaggagctccaggacgccttcatggccaagatccccaatgccgtgctcgggcaggggtacgggatgacggaggcaggccccgtgctggcgatgtgcctggccttcgccaaggagccgtacccggtcaagtccgggtcgtgcggcaccgtggtgcggaacgcggagctgaagatcgtcgaccccgacaccggcgccgccctcggccggaaccagcccggcgagatctgcatccgcggggagcagatcatgaaaggttacctgaacgaccccgagtcgacgaagaacaccatcgaccaggacggctggctgcacaccggcgacatcggctacgtggacgacgacgacgagatcttcatcgtcgacaggctcaaggagatcatcaagtacaagggcttccaggtgccgccggcggagctggaggcgctcctcatcacgcacccggagatcaaggacgccgccgtcgtctcaatgaacgacgaccttgctggtgaaatcccggtcgccttcatcgtgcggaccgaaggttctcaagtcaccgaggatgagatcaagcaattcgtcgccaaggaggtggttttctacaagaagatccacaaggtcttcttcaccgaatccatccccaagaacccgtcgggcaagatcctgaggaaggacttgagagccaggctcgccgccggtgttcactga

SEQ ID NO: 3 - DNA, 1516 bp, Zea mays
ccggcgctcgcgcggctttctttcccaactccgacgaaggctagctacaccaccttgtgcgggctcgtctccatcgcccgccacccgctccgtcgtcgtcgtccccgccgcgccgatcccgaatcgaatggggagcctggcgtccgagaggaaggtggtcgggtgggccgccagggacgccaccggacacctctccccctactcctacaccctcaggaacacaggccctgaagatgtggtggtgaaggtgctctactgcgggatctgccacacggacatccaccaggccaagaaccacctcggggcttcaaagtatcctatggtccctgggcacgaggtggtcggcgaggtggtggaggtcgggcccgaagtggccaagtacggcgtcggcgacgtggtaggcgtcggggtgatcgttgggtgctgccgcgagtgcagcccctgcaaggccaacgttgagcagtactgcaacaagaagatctggtcatacaacgacgtctacactgatggacggcccacgcagggtggattcgcctccaccatggtcgtcgaccagaagtttgtggtgaagatcccggcgggtctggctccggagcaagcggcgccgctgctgtgcgctggcgtgacggtgtacagcccgctgaagcactttgggctgacgaacccgggcctccgtggcggcatcctgggcctcggcggcgtgggccacatgggcgtgaaggtagccaaggccatgggccaccacgtgacggtgatcagctcgtcgtccaagaagcgcgcggaggcaatggaccacctcggcgcggacgcgtacctagtgagctcggacgccgcggccatggcggcggccgccgactcgctggactacatcatcgacacggtgcccgtgcaccacccgctggagccgtacctggcgctgctgaagctggacggcaagctcgtgctgctgggcgtcatcggcgagcccctgagcttcgtgtcgcccatggtgatgctggggcggaaggccatcacggggagcttcatcggcagcatcgacgagaccgctgaggtgcttcagttctgcgtcgacaaggggctcacctcccagatcgaggtggtcaagatggggtacgtgaacgaggcgctggagcggctggagcgcaacgacgtccgctaccgcttcgtcgtcgacgtcgccggtagcaacgtcgaggcggaggcggcggcggcggatgcggccagcaactgatggcaccgcgtcgtcgagtcgaaccacgtctgtgcgccgcgtgcaacgttcgttcgggtcgagtctgcgtgcaacgttctgcttcctttactagttgttgtctttccgccttcttgccgttctgttctgggctttgagatgagacgatggatggtcagtttttaatgtcagactgaataactacgtatagtactgtagtattactcggagtacgccagaatgtggtgtggtgtcagtctcaccagcaatctggatttgccaagtgtttctattttttaaaaaaaa

SEQ ID NO: 4 - DNA, 1311 bp, Zea mays
ctcgtgccccaacgcgctagctagttctattgccgcaccccagatctccaggagggactcgttctttcagctaactacactgcacgcaatggccaccacggcgaccgaggcggcgccggcgcaggagcagcaggccaacggcaacggcgagcagaagacgcggcactccgaggtcggccacaagagcctgctcaagagcgacgacctctaccaggtaaacagagagcacactccggatcctgcctgcccatttccggcctcgcgctctagatcttatcttccgtggctcgaatctgaccgggggaagaatcacttccaccagtacatcctggacacgagcgtgtacccgcgggagccggagagcatgaaggagctccgcgaggtcaccgccaagcacccatggtatgttccgctttcccgggccgatctctcgacggcgacctagctgctggacgacagatcgagatctgaaaacatggcgtgcaggaacctgatgacgacctccgccgacgaggggcagttcctgaacatgctcatcaagctcatcggcgccaagaagaccatggagatcggcgtgtacaccggctactccctcctcgccacggcgctcgccctcccggaggacggcacggtccgtcgttgttccctcccttttcccagatctgccaccccacctccctgaaggcgaagcagctagcggtcctcccactgataaaccaaggattcctctctccctctctttgttgttcgtcccgccccgcagatcttggccatggacatcaaccgcgagaactacgagctgggcctgccctgcatcgagaaggccggcgtcgcccacaagatcgacttccgcgagggtcccgcgctccccgtcctcgacgacctcatcgcggaggtacgaaatggccaggccgccagatctgtgcttcttcgcctatatcgatcgagtagaagttgacatgacatctcggacccgtgtctgtctgcatctgcgcaggagaagaaccacgggtcgttcgacttcgtcttcgtggacgccgacaaggacaactacctcaactaccacgagcggctgctgaagctggtgaagctgggcggcctcatcggctacgacaacacgctgtggaacggctccgtcgtgctccccgacgacgcgcccatgcgcaagtacatccgcttctaccgcgacttcgtgctcgtcctcaacaaggcgctcgccgccgacgaccgcgtcgagatctgccagctccccgtcggcgacggcgtcaccctctgccgccgcgtcaagtgaaaacatgccctggcctggcc

SEQ ID NO: 5 - DNA, 1150 bp, Zea mays
cgcaagccagtgccgcgcccagatctccgcgacagatcagtcgttcgtccagctaactgcactgcactgcactgcacgcaatggccaccacggcgaccgaggcgaccaagacgactgcaccggcgcaggagcagcaggccaacggcaacggcaacggcaacggcgagcagaagacgcgccactccgaggtcggccacaagagcctgctcaagagcgacgacctctaccaggtaaacaagctgggcgcaatgaatggctgaatctgaccgggatctgagtctctgaccgcgggggaagaatgatccgcagtacatcctggacacgagcgtgtacccgcgggagccggagagcatgaaggagctgcgcgagatcaccgccaagcacccatggtatgtcccgctagcttttcgccctgtcgtacgtggtggattcgagtgtgtgggcctgctggacgtggacagaccgagatctgagaacgaacatggcgtggcgtgcaggaacctgatgaccacctccgccgacgagggccagttcctcaacatgctcatcaagctcatcggcgccaagaagaccatggagatcggcgtctacaccggctactcgctcctcgccaccgcgctcgcactcccggaggacggcacggtcggttccctttctctctctctcccagatctgccactgaactgatagaccaaggatctttacccttctctctctcccgcagatcttggccatggacatcaaccgcgagaactacgagctaggcctgccctgcatcaacaaggccggcgtgggccacaagatcgacttccgcgagggccccgcgctccccgtcctggacgacctcgtggcggacaaggagcagcacgggtcgttcgacttcgccttcgtggacgccgacaaggacaactacctcagctaccacgagcggctcctgaagctggtgaggcccggcggcctcatcggctacgacaacacgctgtggaacggctccgtcgtgctccccgacgacgcgcccatgcgcaagtacatccgcttctaccgcgacttcgtcctcgccctcaacagcgcgctcgccgccgacgaccgcgtcgagatctgccagctccccgtcggcgacggcgtcacgctctgccgccgcgtcaagtga

SEQ ID NO: 6 - protein, 371 aa, Zea mays
MTVVDAVVSSTDAGAPAAAATAVPAGNGQTVCVTGAAGYIASWLVKLLLEKGYTVKGTVRNPDDPKNAHLKALDGAAERLILCKADLLDYDAICRAVQGCQGVFHTASPVTDDPEQMVEPAVRGTEYVINAAAEAGTVRRVVFTSSIGAVTMDPKRGPDVVVDESCWSDLEFCEKTRNWYCYGKAVAEHAAWETARRRGVDLVVVNPVLVVGPLLQATVNASIAHILKYLDGSARTFANAVQAYVDVRDVADAHLRVFESPRASGRHLCAERVLHREDVVRILAKLFPEYPVPARCSDEVNPRKQPYKFSNQKLRDLGLQFRPVSQSLYDTVKNLQEKGHLPVLGERTTTEAADKDAPTAEMQQGGIAIRA

SEQ ID NO: 7 - protein, 555 aa, Zea mays
MGSVDAAIAVPVPAAEEKAVEEKAMVFRSKLPDIEIDSSMALHTYCFGKMGEVAERACLIDGLTGASYTYAEVESLSRRAASGLRAMGVGKGDVVMSLLRNCPEFAFTFLGAARLGAATTTANPFYTPHEVHRQAEAAGARLIVTEACAVEKVREFAAERGIPVVTVDGRFDGCVEFAELIAAEELEADADIHPDDVVALPYSSGTTGLPKGVMLTHRSLITSVAQQVDGENPNLYFRKDDVVLCLLPLFHIYSLNSVLLAGLRAGSTIVIMRKFDLGALVDLVRRYVITIAPFVPPIVVEIAKSPRVTAGDLASIRMVMSGAAPMGKELQDAFMAKIPNAVLGQGYGMTEAGPVLAMCLAFAKEPYPVKSGSCGTVVRNAELKIVDPDTGAALGRNQPGEICIRGEQIMKGYLNDPESTKNTIDQDGWLHTGDIGYVDDDDEIFIVDRLKEIIKYKGFQVPPAELEALLITHPEIKDAAVVSMNDDLAGEIPVAFIVRTEGSQVTEDEIKQFVAKEVVFYKKIHKVFFTESIPKNPSGKILRKDLRARLAAGVH

SEQ ID NO: 8 - protein, 367 aa, Zea mays
MGSLASERKVVGWAARDATGHLSPYSYTLRNTGPEDVVVKVLYCGICHTDIHQAKNHLGASKYPMVPGHEVVGEVVEVGPEVAKYGVGDVVGVGVIVGCCRECSPCKANVEQYCNKKIWSYNDVYTDGRPTQGGFASTMVVDQKFVVKIPAGLAPEQAAPLLCAGVTVYSPLKHFGLTNPGLRGGILGLGGVGHMGVKVAKAMGHHVTVISSSSKKRAEAMDHLGADAYLVSSDAAAMAAAADSLDYIIDTVPVHHPLEPYLALLKLDGKLVLLGVIGEPLSFVSPMVMLGRKAITGSFIGSIDETAEVLQFCVDKGLTSQIEVVKMGYVNEALERLERNDVRYRFVVDVAGSNVEAEAAAADAASN

SEQ ID NO: 9 - protein, 258 aa, Zea mays
MATTATEAAPAQEQQANGNGEQKTRHSEVGHKSLLKSDDLYQYILDTSVYPREPESMKELREVTAKHPWNLMTTSADEGQFLNMLIKLIGAKKTMEIGVYTGYSLLATALALPEDGTILAMDINRENYELGLPCIEKAGVAHKIDFREGPALPVLDDLIAEEKNHGSFDFVFVDADKDNYLNYHERLLKLVKLGGLIGYDNTLWNGSVVLPDDAPMRKYIRFYRDFVLVLNKALAADDRVEICQLPVGDGVTLCRRVK

SEQ ID NO: 10 - protein, 266 aa, Zea mays
MATTATEATKTTAPAQEQQANGNGNGNGEQKTRHSEVGHKSLLKSDDLYQYILDTSVYPREPESMKELREITAKHPWNLMTTSADEGQFLNMLIKLIGAKKTMEIGVYTGYSLLATALALPEDGTILAMDINRENYELGLPCINKAGVGHKIDFREGPALPVLDDLVADKEQHGSFDFAFVDADKDNYLSYHERLLKLVRPGGLIGYDNTLWNGSVVLPDDAPMRKYIRFYRDFVLALNSALAADDRVEICQLPVGDGVTLCRRVK

SEQ ID NO: 11 - DNA, 17 bp, Artificial Sequence
  Synthetic
atgagcccagaacgacg

SEQ ID NO: 12 - DNA, 17 bp, Artificial Sequence
  Synthetic
tcagatctcggtgacgg

SEQ ID NO: 13 - DNA, 1667 bp, Zea mays
  misc_feature (603)..(603): n is a, c, g, or t
atgggttccgtagacgcggcgatcgcggtgccggtgccggcggcggaggagaaggcggtggaggagaaggcgatggtgttccggtccaagcttcccgacatcgagatcgacagcagcatggcgctgcacacctactgcttcgggaagatgggcgaggtggcggagcgggcgtgcctgatcgacgggctgacgggcgcgtcgtacacgtacgcggaggtggagtccctgtcccggcgcgccgcgtcggggctgcgcgccatgggggtgggcaagggcgacgtggtgatgagcctgctccgcaactgccccgagttcgccttcaccttcctgggcgccgcccgcctgggcgccgccaccaccacggccaacccgttctacaccccgcacgaggtgcaccgccaggcggaggcggccggcgcccggctcatcgtgaccgaggcctgcgccgtggagaaggtgcgggagttcgcggcggagcggggcatccccgtggtcaccgtcgacgggcgcttcgacggctgcgtggagttcgccgagctgatcgcggccgaggagctggaggctgacgccgacatccaccccgacgacgtcgtcgcgctgccntactcctccggcaccaccgggctgcccaagggcgtcatgctcacccaccgcagcctcatcaccagcgtcgcgcagcaggttgatggcgagaacccgaacctgtacttccgcaaggacgacgtggtgctgtgcctgctgccgctgttccacatctactcgctgaactcggtgctgctggccggcctgcgcgcgggctccaccatcgtgatcatgcgcaagttcgacctgggcgcgctggttgacctggtgcgcaggtacgtgatcaccatcgcgcccttcgtgccgcccatcgtggtggagatcgccaagagcccccgcgtgaccgccggcgacctcgcgtccatccgcatggtcatgtccggcgccgcgcccatgggcaaggagctccaggacgccttcatggccaagatccccaatgccgtgctcgggcaggggtacgggatgacggaggcaggccccgtgctggcgatgtgcctggccttcgccaaggagccgtacccggtcaagccgggtcgtgcggcaccgtggtgcggaacgcggagctgaagatcgtcgaccccgacaccggcgccgccctcggccggaaccagcccggcgagatctgcatccgcggggagcagatcatgaaaggttacctgaacgaccccgagtcgacgaagaacaccatcgaccaggacggctggctgcacaccggcgacatcggctacgtggacgacgacgacgagatcttcatcgtcgacaggctcaaggagatcatcaagtacaagggcttccaggtgccgccggcggagctggaggcgctcctcatcacgcacccggagatcaaggacgccgccgtcgtctcaatgaacgacgaccttgctggtgaaatcccggtcgccttcatcgtgcggaccgaaggttctcaagtcaccgaggatgagatcaagcaattcgtcgccaaggaggtggttttctacaagaagatccacaaggtcttcttcaccgaatccatccccaagaacccgtcgggcaagatcctgaggaaggacttgagagccaggctcgccgccggtgttcactga

SEQ ID NO: 14 - DNA, 19 bp, Zea mays
atgagcctgctccgcaact

SEQ ID NO: 15 - DNA, 21 bp, Zea mays
ccgagttcagcgagtagatgt

SEQ ID NO: 16 - DNA, 487 bp, Zea mays
  misc_feature (318)..(318): n is a, c, g, or t
atgagcctgctccgcaactgccccgagttcgccttcaccttcctgggcgccgcccgcctgggcgccgccaccaccacggccaacccgttctacaccccgcacgaggtgcaccgccaggcggaggcggccggcgcccggctcatcgtgaccgaggcctgcgccgtggagaaggtgcgggagttcgcggcggagcggggcatccccgtggtcaccgtcgacgggcgcttcgacggctgcgtggagttcgccgagctgatcgcggccgaggagctggaggctgacgccgacatccaccccgacgacgtcgtcgcgctgccntactcctccggcaccaccgggctgcccaagggcgtcatgctcacccaccgcagcctcatcaccagcgtcgcgcagcaggttgatggcgagaacccgaacctgtacttccgcaaggacgacgtggtgctgtgcctgctgccgctgttccacatctactcgctgaactcgg

SEQ ID NO: 17 - DNA, 1398 bp, Zea mays
ggcacgagaggacacaagcgagcgctagccagaagagcagctgcaggtactattatcatcgtcgtcgtcgtcgccaggatgaccgtcgtcgacgccgtcgtctcctccaccgatgccggcgcccctgccgccgccgccgcaccggtaccggcggggaacgggcagaccgtgtgcgtcaccggcgcggccgggtacatcgcctcgtggttggtgaagctgctgctcgagaagggatacactgtgaagggcaccgtgaggaacccagatgacccgaagaacgcgcacctcagggcgctggacggcgccgccgagcggctgatcctctgcaaggccgatctgctggactacgacgccatctgccgcgccgtgcagggctgccagggcgtcttccacaccgcctcccccgtcaccgacgacccggagcaaatggtggagccggcggtgcgcggcaccgagtacgtgatcaacgcggcggcggaggccggcacggtgcggcgggtggtgttcacgtcgtccatcggcgccgtgaccatggaccccaagcgcgggcccgacgtcgtggtcgacgagtcgtgctggagcgacctcgagttctgcgagaaaaccaggaactggtactgctacggcaaggcggtggcggagcaggcggcgtgggaggcggcccggcggcggggcgtggacctggtggtggtgaaccccgtgctggtggtgggccccctgctgcaggcgacggtgaacgccagcatcgcgcacatcctcaagtacctggacggctcggcccgcaccttcgccaacgccgtgcaggcgtacgtggacgtgcgcgacgtggccgacgcgcacctccgcgtcttcgagagcccccgcgcgtccggccgccacctctgcgccgagcgcgtcctccaccgcgaggacgtcgtccgcatcctcgccaagctcttccccgagtaccccgtcccagccaggtgctccgacgaggtgaatccgcggaagcagccgtacaagttctccaaccagaagctccgggacctggggctgcagttccggccggtcagccagtcgctttacgacacggtgaagaacctccaggagaagggccacctgccggtgctcggagagcggacgacgacggaggccgccgacaaggatgcccccgcggccgagatgcagcagggagggatcgccatccgtgcctgagagggcgatgccacacatgaacacaaagcaatgttcatactgctgccctgcacctgcaccttcccctgctgtgtaaacaggcctgtgtttgttctggctgatagtgatgtaccctaagacttgtaacgtcatgttcgttcttgtgaactatagcgagtgaataaaattggttaatgttggatattcaaaaaaaaaaaaaaaaaa

SEQ ID NO: 18 - DNA, 24 bp, Zea mays
agaagggatacactgtgaagggca

SEQ ID NO: 19 - DNA, 21 bp, Zea mays
cgtagcagtaccagttcctgg

SEQ ID NO: 20 - DNA, 617 bp, Zea mays
aggacacaagcgagcgctagccagaagagcagctgcaggtactattatcatcgtcgtcgtcgtcgccaggatgaccgtcgtcgacgccgtcgtctcctccaccgatgccggcgcccctgccgccgccgccgcaccggtaccggcggggaacgggcagaccgtgtgcgtcaccggcgcggccgggtacatcgcctcgtggttggtgaagctgctgctcgagaagggatacactgtgaagggcaccgtgaggaacccagatgacccgaagaacgcgcacctcagggcgctggacggcgccgccgagcggctgatcctctgcaaggccgatctgctggactacgacgccatctgccgcgccgtgcagggctgccagggcgtcttccacaccgcctcccccgtcaccgacgacccggagcaaatggtggagccggcggtgcgcggcaccgagtacgtgatcaacgcggcggcggaggccggcacggtgcggcgggtggtgttcacgtcgtccatcggcgccgtgaccatggaccccaagcgcgggcccgacgtcgtggtcgacgagtcgtgctggagcgacctcgagttctgcgagaaaaccaggaactggtactgctacg

SEQ ID NO: 21 - DNA, 1516 bp, Zea mays; identical to SEQ ID NO: 3

SEQ ID NO: 22 - DNA, 24 bp, Zea mays
aagatgtggtggtgaaggtgctct

SEQ ID NO: 23 - DNA, 24 bp, Zea mays
tgtcgatgatgtagtccagcgagt

SEQ ID NO: 24 - DNA, 648 bp, Zea mays
aagatgtggtggtgaaggtgctctactgcgggatctgccacacggacatccaccaggccaagaaccacctcggggcttcaaagtatcctatggtccctgggcacgaggtggtcggcgaggtggtggaggtcgggcccgaagtggccaagtacggcgtcggcgacgtggtaggcgtcggggtgatcgttgggtgctgccgcgagtgcagcccctgcaaggccaacgttgagcagtactgcaacaagaagatctggtcatacaacgacgtctacactgatggacggcccacgcagggtggattcgcctccaccatggtcgtcgaccagaagtttgtggtgaagatcccggcgggtctggctccggagcaagcggcgccgctgctgtgcgctggcgtgacggtgtacagcccgctgaagcactttgggctgacgaacccgggcctccgtggcggcatcctgggcctcggcggcgtgggccacatgggcgtgaaggtagccaaggccatgggccaccacgtgacggtgatcagctcgtcgtccaagaagcgcgcggaggcaatggaccacctcggcgcggacgcgtacctagtgagctcggacgccgcggccatggcggcggccgccgactcgctggactacatcatcgaca

SEQ ID NO: 25 - DNA, 4630 bp, Artificial Sequence
  Synthetic
catggatccaggagcggccgcagatctccaataagagctctgatctcataagcccgatggctactaagttttactatttaccaagacttttgaatattaaccttcttgtaacgagtcggttaaatttgattgtttagggttttgtattatttttttttggtcttttaattcatcactttaattccctaattgtctgttcatttcgttgtttgtttccggatcgataatgaaatgtaagagatatcatatataaataataaattgtcgtttcatatttgcaatcttttttttacaaacctttaatcgttgtatgtatgacattttcttcttgttatattagggggaaataatgttaaataaaagtacaaaataaactacagtacatcgtactgaataaattacctagccaaaaagtacacctttccatatacttcctacatgaaggcattttcaacattttcaaataaggaatgctacaaccgcataataacatccacaaatttttttataaaataacatgtcagacagtgattgaaagattttattatagtttcgttatcttcttttctcattaagcgaatcactacctaacacgtcattttgtgaaatattttttgaatgtttttatatagttgtagcattcctcttttcaaattagggtttgtttgagatagcatttcagccggttcatacaacttaaaagcatactctaatgctggaaaaaagactaaaaaatcttgtaagttagcgcagaatattgacccaaattatatacacacatgaccccatatagagactaattacacttttaaccactaataattattactgtattataacatctactaattaaacttgtgagtttttgctagaattattatcatatatactaaaaggcaggaacgcaaacattgccccggtactgtagcaactacggtagacgcattaattgtctatagtggacgcattaattaacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcgccaagcttagacaaacaccccttgttatacaaagaatttcgctttacaaaatcaaattcgagaaaataatatatgcactaaataagatcattcggatctaatctaaccaattacgatacgctttgggtacacttgatttttgtttcagtggttacatatatcttgttttatatgctatctttaaggatctgcacaaagattatttgttgatgttcttgatggggctcagaagatttgatatgatacactctaatctttaggagataccagccaggattatattcagtaagacaatcaaattttacgtgttcaaactcgttatcttttcattcaaaggatgagccagaatctttatagaatgattgcaatcgagaatatgttcggccgatatgcctttgttggcttcaatattctacatatcacacaagaatcgaccgtattgtaccctctttccataaaggaaaacacaatatgcagatgcttttttcccacatgcagtaacatataggtattcaaaaatggctaaaagaagttggataacaaattgacaactatttccatttctgttatataaatttcacaacacacaaaagcccgtaatcaagagtctgcccatgtacgaaataacttctattatttggtattgggcctaagcccagctcagagtacgtgggggtaccacatataggaaggtaacaaaatactgcaagatagccccataacgtaccagcctctccttaccacgaagagataagatataagacccaccctgccacgtgtcacatcgtcatggtggttaatgataagggattacatccttctatgtttgtggacatgatgcatgtaatgtcatgagccacaagatccaatggccacaggaacgtaagaatgtagatagatttgattttgtccgttagatagcaaacaacattataaaaggtgtgtatcaataggaactaattcactcattggattcatagaagtccattcctcctaagtatctagaaac